SEQUENCE LISTING (19 sequences)

SEQ ID NO: 1 - DNA, 20 bp, Artificial
  PCR primer
cttgtcgctgtcttgcactc

SEQ ID NO: 2 - DNA, 20 bp, Artificial
  PCR primer
aatctgtcaggctggtctgc

SEQ ID NO: 3 - DNA, 20 bp, Artificial
  PCR primer
aaagatgagtatgcctgccg

SEQ ID NO: 4 - DNA, 23 bp, Artificial
  PCR primer
cctccatgatgatgctgcttaca

SEQ ID NO: 5 - DNA, 19 bp, Artificial
  PCR primer
tccaaagcgtcttcaccag

SEQ ID NO: 6 - DNA, 21 bp, Artificial
  PCR primer
gagacagcctttgttcctgag

SEQ ID NO: 7 - DNA, 19 bp, Artificial
  PCR primer
cctgcacacactgatgcac

SEQ ID NO: 8 - DNA, 20 bp, Artificial
  PCR primer
gtctcgaattttggcgaagt

SEQ ID NO: 9 - DNA, 18 bp, Artificial
  PCR primer
acgcctcaaggagctggt

SEQ ID NO: 10 - DNA, 19 bp, Artificial
  PCR primer
cgcttcagcgacacaagat

SEQ ID NO: 11 - DNA, 20 bp, Artificial
  PCR primer
tgcaaccgacgattcttcta

SEQ ID NO: 12 - DNA, 20 bp, Artificial
  PCR primer
cgagctgtttacgtttgacg

SEQ ID NO: 13 - protein, 6 aa, Artificial
  Polypeptide sequence derived from collagen
KGGCLK

SEQ ID NO: 14 - protein, 13 aa, Artificial
  Polypeptide sequence derived from collagen
STQNASLLSLTVC

SEQ ID NO: 15 - protein, 12 aa, Artificial
  Polypeptide sequence derived from collagen
CLKQNGGNFSLG

SEQ ID NO: 16 - protein, 12 aa, Artificial
  Polypeptide sequence derived from collagen
SLKQNGGNFSLC

SEQ ID NO: 17 - protein, 17 aa, Artificial
  Polypeptide sequence derived from collagen
KGGCLKQNGGNFSLGGK

SEQ ID NO: 18 - protein, 19 aa, Artificial
  Polypeptide sequence derived from collagen
KGGCSTQNAQLLSLIVGKA

SEQ ID NO: 19 - protein, 18 aa, Artificial
  Polypeptide sequence derived from collagen
KGGSTQNAQLLSLIVGKA

(19) United States
(12) Patent Application Publication
(10) Pub. No.: US 2006/0240002 A1
(43) Pub. Date: Oct. 26, 2006

(54) Methods of inhibiting angiogenesis-dependent conditions mediated by cryptic epitopes of extracellular matrix components

(76) Inventors: Peter C. Brooks, Carmel, NY (US); Abebe Akalu, Forest Hills, NY (US); Alexandra Cretu, Wilmington, DE (US); Desiree Policarpio, Douglaston, NY (US)

Correspondence Address: WILSON SONSINI GOODRICH & ROSATI, 650 PAGE MILL ROAD, PALO ALTO, CA, 94304-1050, US

(21) Appl. No.: 11/371,626

(22) Filed: Mar. 9, 2006

Related U.S. Application Data
(60) Provisional application No. 60/711,177, filed on Aug. 25, 2005.
(60) Provisional application No. 60/711,049, filed on Aug. 24, 2005.
(60) Provisional application No. 60/660,713, filed on Mar. 11, 2005.

(51) Int. Cl. A61K 39/395; C12Q 1/68; G01N 33/567; C07K 16/44

(52) U.S. Cl. 424/141.1; 435/6; 435/7.2; 530/388.15

(57) ABSTRACT

The invention provides methods for identifying genes and proteins modulated by an antagonist to a cryptic epitope of an ECM component that specifically binds to the ECM component. It additionally provides methods for using the products of the identified genes, or for using the identified proteins, for inhibiting angiogenesis, tumor metastasis, and other tumor developmental processes, including cell migration, cell adhesion, cell proliferation, and tumor growth and for treating angiogenesis-dependent conditions. The present invention also relates to antagonists of cryptic epitopes of ECM components, wherein binding of these antagonists to cryptic epitopes of ECM components results in modulation of the expression of IGFBP-4, TSP-1, Id-1, p27^{KIP} or p21^{CIP}, and methods of using these antagonists for inhibiting angiogenesis, tumor metastasis, and other tumor development processes as well as for treating angiogenesis-dependent conditions.

### RELATED APPLICATIONS

This application claims the benefit of U.S. Provisional Application No. 60/711,177, entitled “METHODS OF INHIBITING ANGIOGENESIS-DEPENDENT CONDITIONS MEDIATED BY CRYPTIC EPITOPES OF EXTRACELLULAR MATRIX COMPONENTS,” filed Aug. 25, 2005, by Peter Brooks et al., U.S. Provisional Application No. 60/711,049, entitled “METHODS OF INHIBITING ANGIOGENESIS-DEPENDENT CONDITIONS MEDIATED BY CRYPTIC EPITOPES OF EXTRACELLULAR MATRIX COMPONENTS,” filed Aug. 24, 2005, by Peter Brooks et al., and U.S. Provisional Application No. 60/660,713, entitled “METHODS OF INHIBITING ANGIOGENESIS-DEPENDENT CONDITIONS MEDIATED BY CRYPTIC EPITOPES OF EXTRACELLULAR MATRIX COMPONENTS,” filed Mar. 11, 2005, by Peter Brooks et al., each of which is incorporated herein by reference in its entirety.

### REFERENCE TO GOVERNMENT GRANT

This invention was made, in part, with government support under NIH RO1 CA91645, awarded by the National Institutes of Health. The government has rights to the invention.

### FIELD OF THE INVENTION

The present invention relates to the field of medicine, specifically to methods and compositions for inhibiting angiogenesis and other processes important in tumor metastasis, based on identifying genes that are modulated by inhibition of cryptic epitopes of extracellular matrix components.

### BACKGROUND OF THE INVENTION

The effective treatment of malignant tumors is impeded by the development of resistance to standard therapeutic modalities as well as metastatic dissemination of tumor cells. Metastasis, or the spread of malignant tumor cells from the primary tumor mass to distant sites, involves a complex series of interconnected events. Understanding the biochemical, molecular, and cellular processes that regulate tumor metastasis are of great importance to treating these tumors. The metastatic cascade is thought to be initiated by a series of biochemical and genetic alterations leading to changes in cell-cell interactions allowing disassociation of cells from the primary tumor mass. These events are followed by local invasion and migration through the proteolytically-remodeled extracellular matrix (ECM) to allow access of the tumor cells to the host circulation. In order to establish secondary metastatic deposits, the malignant cells evade the host immune surveillance, arrest in the microvasculature and extravasate out of the circulation. Finally, circulating tumor cells can adhere to the ECM in a new location, proliferate, and recruit new blood vessels by induction of angiogenesis, thereby forming secondary metastatic foci (Liotta, et al., Cell 1991, 64:327-336; Wyckoff, et al., Cancer Res. 2000, 60:2504-2511; Kurschat, et al., Clin. Exp. Dermatol. 2000, 25:482-489; Pantel, et al., Nat. Rev. Cancer 2004, 4:448-456; Hynes, et al., Cell 2003, 113:821-823; Bashyam, M. D., Cancer 2002, 94:1821-1829).

Identification of proteins involved in tumor cell interactions with the proteolytically-remodeled ECM can provide novel therapeutic targets and treatment strategies for treating malignant tumors. While many studies have confirmed the importance of targeting specific secreted growth factors, proteases, cell surface adhesion receptors and intracellular regulatory molecules, the success of these approaches has been limited due in part to the genetic instability of tumor cells (Molife, et al., Crit. Rev. Oncol. Hematol. 2002, 44:81-102; Brown, et al., Melanoma 2001, 3:344-352; Soengas, et al., Oncogene 2003, 22:3138-3151; Masters, et al., Nat. Rev. Cancer 2003, 3:517-525). Therefore, identifying new functional targets within the non-cellular compartment provides a promising clinical strategy.

The ECM is an interconnected molecular network that not only provides mechanical support for cells and tissues, but also regulates biochemical and cellular processes such as adhesion, migration, gene expression and differentiation. Extracellular matrix components include, e.g., collagen, fibronectin, osteopontin, laminin, fibrinogen, elastin, thrombospondin, tenascin, and vitronectin. Studies have identified cryptic sites, including HUIV26, within the collagen, that regulate angiogenesis and endothelial cell behavior (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210). This functional cryptic site was shown to be highly expressed within the ECM of malignant tumors and within the sub-endothelial basement membrane of tumor-associated blood vessels, and its exposure found to be involved in the regulation of angiogenesis in vivo (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210, and U.S. Ser. No. 09/478,977, now U.S. Pub. No. 2003/0113331, the disclosure of which is incorporated herein by reference in its entirety).

Cryptic sites in the ECM component, laminin, have also been described, e.g., in U.S. Publication No. 2004/224896 A1, “STQ Peptides,” to Brooks, et al. (the disclosure of which is incorporated herein by reference in its entirety), and WO 2004/087734, “STQ Peptides,” to Brooks, et al. These publications describe peptide antagonists selective for denatured laminin that inhibit angiogenesis, tumor growth and metastasis. Antibodies selective for denatured laminin have also been identified.

There are potentially important cryptic epitopes in other ECM proteins, e.g., fibronectin (Hocking, et al., J. Cell. Biol. 2002, 158:175-184), fibrinogen (Medved et al., Ann. N.Y. Acad. Sci. 2001, 936:185-204), and osteopontin (Yamamoto, et al., J. Clin. Invest. 2003, 112:181-188).

Angiogenesis is the physiological process by which new blood vessels develop from pre-existing vessels (Varner, et al., Cell Adh. Commun. 1995, 3:367-374; Blood, et. al., Biochim. Biophys. Acta. 1990, 1032:89-118; Weidner, et al., J. Natl. Cancer Inst. 1992, 84:1875-1887). Angiogenesis has been suggested to play roles in both normal and pathological processes. For example, angiogenic processes are involved in the development of the vascular systems of animal organs and tissues. They are also involved in transitory phases of angiogenesis, for example during the menstrual cycle, in pregnancy, and in wound healing. On the other hand, a number of diseases are known to be associated with deregulated angiogenesis.

In certain pathological conditions, angiogenesis is recruited as a means to provide adequate blood and nutrient supply to the cells within the affected tissue. Many of these pathological conditions involve abberant cell proliferation or regulation. Therefore, inhibition of angiogenesis is a potentially useful approach to treating diseases that are characterized by unregulated blood vessel development. For example, angiogenesis is involved in pathologic conditions including: ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; vitamin A deficiency; Sjorgen's disease; acne rosacea; mycobacterium infections; bacterial and fungal ulcers; Herpes simplex infections; systemic lupus; retrolental fibroplasia; rubeosis; capillary proliferation in atherosclerotic plaques, and; osteoporosis. Angiogenesis is also involved in cancer-associated disorders, including, for example, solid tumors, tumor metastases, blood borne tumors such as leukemias, angiofibromas, Kaposi's sarcoma, benign tumors such as hemangiomas, acoustic neuromas, neurofibromas, trachomas, and pyogenic granulomas, as well as other cancers which require neovascularization to support tumor growth. Other angiogenesis-dependent conditions include, for example, hereditary diseases such as Osler-Weber Rendu disease and haemorrhagic teleangiectasia; myocardial angiogenesis; plaque neovascularization; hemophiliac joints and wound granulation. Progression of tumors such as melanoma, from benign to metastatic disease, correlates with an increase in angiogenesis as well as an increase in expression of specific cell adhesion receptors including integrins (Srivastava, et al., Am. J. Pathol. 1988, 133:419-423; Koth, et al., N. Engl. J. Med. 1991, 325: 171-182). Thus, angiogenesis likely plays a critical role in melanoma progression.

Examples of normal physiological processes involving angiogenesis include embryo implantation, embryogenesis and development, and wound healing. It is conceivable that angiogenesis can also be altered to beneficially influence normal physiological processes. Furthermore, studies have indicated that adipose tissue growth is dependent on angiogenesis, likely due to the need for recruitment of new blood vessels. Delivery of an angiogenesis inhibitor to mice was found to reduce diet-induced obesity, the most common type of obesity in humans (Brakenhielm, et al., Circ. Res. 2004, 94(12):1579-88). This finding suggests a utility for angiogenesis inhibitors in addressing obesity and certain related conditions. Therefore, the inhibition of angiogenesis potentially can be applied in normal angiogenic responses where a prophylactic or therapeutic need or benefit exists.

Molecular alterations that occur in both tumor and stromal cells are thought to potentiate angiogenesis in part by modifying expression and bioavailability of angiogenic growth factors as well as altering expression of matrix-degrading proteases. Collectively, these and other molecular changes help to create a microenvironment conducive to new blood vessel growth, one factor that contributes to metastasis and tumor growth. There is evidence for the importance of numerous molecular regulators that contribute to new blood vessel growth, including matrix-degrading proteases such as MMP-9, angiogenesis inhibitors such as TSP-1 and angiogenic growth factors such as VEGF (see, e.g., Yu, et al., Proc. Natl. Acad. Sci. USA 1999, 96:14517-14522 and Dameron, et al., Science 1994, 265:1582-1584). These molecular regulators, the proteins that in turn regulate them, and any of a number of other molecules potentially affect angiogenesis and metastasis. However, the exact mechanisms of the regulation of these and related processes, including the genes and gene expression patterns involved, have not been determined.

Proteolytic activity plays a crucial role in controlling angiogenesis by releasing matrix-sequestered growth factors as well as remodeling ECM proteins. ECM remodeling results in the exposure of cryptic epitopes, such as the HUIV26 collagen site and sites within laminin. The HUIV26 cryptic collagen epitope is recognized by αvβ3 integrin, which is expressed in tumors.

It has been reported that tumor cell expression of a number of cell-cycle control proteins is influenced by integrins (Zhong, et al., Proc. Natl. Acad. Sci. USA 2000, 97:10026-10031; Stromblad, et al., J. Clin. Invest. 1996, 98:426-433; Clarke, et al., J. Biol. Chem. 1995, 270:22673-22676; Liang, et al., FEBS Letters 2004, 558:107-113). Particular roles that these proteins play in tumor development have not been identified. Integrins appear to influence the cyclin-dependent kinase inhibitors P21^{CIP1} and P27^{KIP1} but a direct relationship between binding of an integrin to an ECM cryptic epitope, and expression of the gene encoding P21^{CIP1} or P27^{KIP1} has not been reported.

Other proteins appear to be involved in integrin signaling, for example, Insulin Growth Factor Binding Proteins (IGFBPs). IGFBPs are a family of secreted proteins that function to regulate IGF-signaling by binding to IGFs, thereby disrupting IGF receptor binding and subsequent signaling (Pollak, et al., Nat. Rev. Cancer 2004, 4:505-518; Mohan, et al., J. Endocrinol. 2002, 175:19-31; LeRoith, et al., Cancer Lett. 2003, 195:127-137). Specific IGFBPs may directly bind to integrin receptors, thereby modulating their function independently from IGFs (McCaig, et al., J. Cell Sci. 2002, 115:4293-4303; Schutt, et al., J. Mol. Endocrinol. 2004, 32:859-868; Furstenberger, et al., Lancet. 2002, 3:298-302). IGFBPs may regulate cellular adhesion, migration and tumor growth by both IGF-dependent and independent mechanisms (McCaig, et al., J. Cell Sci. 2002, 115:4293-4303; Schutt, et al., J. Mol. Endocrinol. 2004, 32:859-868; Furstenberger, et al., Lancet. 2002, 3:298-302). However, regulation of these cellular processes by integrin-receptor binding of IGFBPs, and the exact role of IGFBPs in these processes, have not been established.

Further, the protein Id-1 has been reported to repress TSP-1 expression and regulate angiogenesis in vivo (Volpert, et al., Cancer Cell 2002, 2(6):473-83). P53, a tumor-suppressor protein, has also been reported to play an important role in controlling expression of proteins known to regulate angiogenesis, including VEGF and thrombospondin-1 (TSP-1) (Yu, et al., Proc. Natl. Acad. Sci. USA 1999, 96:14517-14522; Dameron, et al., Science 1994, 265:1582-1584). The p53 status of tumors is believed to impact the efficacy of anti-angiogenic, chemotherapeutic and radiation therapy for the treatment of malignant tumors (Yu, et al., Science 2002, 295:1526-1528; Martin, et al., Cancer Res. 1999, 59:1391-1399; Fridman, et al., Oncogene (2003) 22:9030-9040; Gudkov, et al., Nat. Rev. Cancer 2003, 3:117-128). Despite the possibility that these and other proteins are involved in the integrin-mediated regulation of tumor development processes, e.g., angiogenesis, metastasis, cell adhesion, cell migration, cell proliferation, and tumor growth, the regulation of specific genes in response to cryptic epitopes of ECM component binding has not been previously characterized. This invention identifies the connection between cryptic epitope ECM component binding and the regulation of genes involved in tumor development processes.

### SUMMARY OF THE INVENTION

The present invention relates to methods for the identification of at least one gene or protein, wherein the expression of said gene or protein is modulated by specific binding of an antagonist to a cryptic epitope of an ECM component. It further relates to methods for inhibiting angiogenesis, tumor metastasis, and related processes, including cell migration, cell adhesion, tumor growth, and for treating angiogenesis-dependent conditions using proteins identified based on the modulation of their expression when an antagonist of a cryptic epitope of an ECM component specifically binds to that epitope. The present invention also relates to antagonists of cryptic epitopes of ECM components, wherein binding of the antagonists to the ECM cryptic epitopes results in modulation of the expression of a gene selected from the group of IGFBP-4, TSP-1, Id-1, p27^{KIP1} or p21^{CIP}. Further, the invention includes methods for the use of these antagonists to inhibit angiogenesis, metastasis, and related processes, as well as for treatment of angiogenesis-dependent conditions, and methods for detecting the inhibition of these processes and conditions based on modulation of IGFBP-4, TSP-1, Id-1, p27^{KIP1} or p21^{CIP}. The present invention also contemplates methods of diagnosing an angiogenesis-dependent condition wherein modulation of genes identified according to the identifying methods of the invention is indicative of the presence or severity of the condition.

In particular, the present invention relates to a method for identifying at least one gene or protein, wherein the expression of said gene or protein is modulated by binding of an antagonist to a cryptic epitope of an ECM component, wherein said antagonist specifically binds to said cryptic epitope of said ECM component, comprising the steps of: a) treating cells with the antagonist in the presence of cryptic epitopes of ECM components; b) measuring gene expression or protein levels in the cells; c) comparing said gene expression or protein levels with control gene expression or protein levels measured in cells not treated with the antagonist, and; d) identifying a gene or protein wherein levels in the cells treated with the antagonist are modulated as compared to the control cell gene expression or protein levels.

The invention also relates to methods for inhibiting tumor metastasis, cell adhesion, cell migration, tumor growth, angiogenesis, and for treating angiogenesis-dependent conditions, comprising administering a product of a gene, or a protein, wherein the gene or the protein is modulated by the binding of an antagonist to a cryptic epitope of an ECM component, and wherein said antagonist specifically binds to said cryptic epitope of said ECM component, and wherein the gene or the protein is identified using a method of identifying at least one gene or protein modulated by the binding of said antagonist to said epitope, said method of identifying comprising the steps of: a) treating cells with the antagonist in the presence of cryptic epitopes of ECM components; b) measuring gene expression or protein levels in the cells; c) comparing said gene expression or protein levels with control gene expression or protein levels measured in cells not treated with the antagonist, and; d) identifying a gene or protein wherein levels of the gene or protein in the cells treated with the antagonist are modulated as compared to the control cell gene expression or protein levels.

In other embodiments of the invention, at least two genes or proteins are identified in the method of identifying, and at least one of the at least two genes or proteins identified is IGFBP-4, TSP-1, Id-1, p27^{KIP1} or p21^{CIP}.

The invention also relates to antagonists that specifically bind to a cryptic epitope of an ECM component, wherein binding of said antagonist to said cryptic epitope of said ECM component results in modulation of IGFBP-4, TSP-1, Id-1, p27^{KIP1} or p21^{CIP}. In embodiments, these antagonists are used in methods of inhibiting tumor metastasis, cell adhesion, cell migration, tumor growth, angiogenesis, and in methods for treating angiogenesis-dependent conditions. In embodiments of these methods, the antagonist is administered in conjunction with another antagonist that binds to a cryptic epitope of an ECM component, chemotherapy, radiation therapy, or in conjunction with a cytostatic agent.

In certain embodiments, the antagonist used in the methods of the invention is an antibody or an antibody fragment, for example, a monoclonal antibody, a polyclonal antibody, or in particular, the antagonist is monoclonal antibody HUIV26. In other embodiments, the antagonist used in the methods of the invention is a peptide. In particular embodiments, the antagonist is CLK-peptide, SLK-peptide, KGGCLK-peptide (SEQ ID NO: 13), the peptide NH₂—S-T-Q-N-A-S-L-L-S-L-T-V—C—COOH (SEQ ID NO: 14), STQ-peptide, or STQ-peptide-S.

The present invention further relates to methods for detecting the inhibition of tumor metastasis, cell adhesion, cell migration, tumor growth, and angiogenesis using an antagonist that specifically binds to a cryptic epitope of an ECM component, comprising: measuring the level of IGFBP-4, TSP-1, Id-1, p27^{KIP1} or p21^{CIP}, wherein said level of IGFBP-4, TSP-1, Id-1, p27^{KIP1} or p21^{CIP} is modulated.

### BRIEF DESCRIPTION OF THE DRAWINGS

FIG. 1 Effect of Mab HUIV26 on Adhesion of B16F10 Melanoma Cells to Denatured Collagen Type-IV. To examine the effects of a function blocking Mab directed to a cryptic collagen site has on tumor cell adhesion, in vitro adhesion assays were performed. Non-tissue culture 48-well plates were coated (10.0 μg/ml) with denatured collagen type-IV. Tumor cells (B16F10 melanoma) were resuspended in adhesion buffer in the presence (100 μg/ml) or absence of Mab HUIV26 or an isotype-matched control antibody, and cell adhesion was quantified. Data bars represent mean tumor cell adhesion+standard deviations from triplicate wells. Experiments were completed 3 times with similar results. As described in Example I, Mab HUIV26 specifically inhibited adhesion of B16F10 cells to denatured collagen type-IV as compared to either no treatment (NT) or treatment with an isotype matched control antibody (Control).

FIG. 2 Effect of Mab HUIV26 on Adhesion of 4T1 Breast Carcinoma Cells to Denatured Collagen Type-IV. As described in Example I, Mab HUIV26 specifically inhibited adhesion of 4T1 cells to denatured collagen type-IV as compared to either no treatment (NT) or treatment with an isotype matched control antibody (Control).

FIG. 3 Effect of Mab HUIV26 on Migration of B16F10 Melanoma Cells on Denatured Collagen Type-IV. To examine the effects of a function blocking Mab directed to a cryptic collagen site on tumor cell migration, in vitro migration assays were performed. Membranes from 24-well transwell migration chambers were coated (10.0 μg/ml) with denatured collagen type-IV. Tumor cells (B16F10 melanoma) were resuspended in migration buffer in the presence (100 μg/ml) or absence of Mab HUIV26 of an isotype matched control antibody and seeded into the upper wells of the chambers and migration was quantified following a 4 hour incubation period. Data bars represent mean tumor cell migration+standard errors from triplicate wells as measured by direct cell counts. Experiments were completed 3 times with similar results. As described in Example II, Mab HUIV26 inhibited migration of B16F10 tumor cells as compared to either no treatment (NT) or treatment with an isotype-matched control antibody (Control).

FIG. 4 Metastasis of Injected B16F10 Melanoma Cells Results in the Formation of Melanotic Lesions. Chick embryos were injected intravenously with B16F10 melanoma cells to assess their capacity to colonize the lungs. The figure shows representative examples of 19-day-old chick lungs from either untreated or B16F10 cell injected embryos. As described in Example III, intravenous injections of increasing concentrations of B16F10 melanoma cells resulted in the dose-dependent formation of numerous discrete melanotic lesions, which could readily be seen on the surface of the chick lungs.

FIG. 5 Quantification of Experimental Metastasis. Quantification of dose-dependent B16F10 experimental metastasis. Embryos were allowed to incubate for 7 days, at which time they were sacrificed, lungs removed and the number of pigmented lung tumor lesions quantified. Data bars represent the mean number of tumor lesions per lung, per experimental condition+standard error. Experiments were completed 3 times with similar results. As described in Example III, to quantify the experimental metastasis, the chick lungs were removed and the total number of discrete independent foci was counted on both lobes for each lung and metastasis was expressed as the mean number of discrete B16F10 foci per lung per group.

FIG. 6 Tumor Cells in Chick Lungs. The figure shows frozen sections of lung tissue from either untreated (NT) or B16F10 injected embryos stained by hematoxylin and eosin. Note large tumor cells with irregular nuclei. Photos were taken at 400× magnification. As described in Example III, histological analysis of sections from either normal lungs or lungs from embryos injected with B16F10 cells were stained with hematoxylin and eosin.

FIG. 7 Immunological Confirmation of the Presence of the Melanoma Cells. The figure shows immunofluoresence analysis of the expression of MART-1 antigen within either lung tissue from untreated (NT) or B16F10-injected embryos. Red color indicates expression of the melanoma associated antigen MART-1. Photographs were taken at a magnification of 600× with oil immersion. As described in Example III, lung sections were analyzed for the expression of the melanoma-associated antigen MART-1. No specific expression of the MART-1 antigen was detected in the lungs from untreated control embryos (left panel). In contrast, tumor cells within the lungs derived from embryos injected with the B16F10 cells stained positive for the MART-1 antigen (right panel).

FIG. 8 Effect of Injection of B16F10 Melanoma Cells on Metastasis. To evaluate whether Mab HUIV26 impacts tumor cell metastasis, B16F10 experimental metastasis was examined in the chick embryo model. Chick embryos were injected with B16F10 cells in the presence or absence of Mab HUIV26 or an isotype matched control antibody (1.0 to 100.0 μg/embryo). Embryos were allowed to incubate for 7 days, at which time they were sacrificed, lungs removed and the number of pigmented lung tumor lesions quantified. The figure shows representative examples of 19-day-old chick lungs from each experimental condition. Experiments were completed 3 times with similar results. As described in Example III, injection of untreated B16F10 melanoma cells resulted in the formation of extensive lung foci. In contrast, lungs from chick embryos treated with Mab HUIV26 exhibited a dramatic reduction in B16F10 lung surface lesions. Histological examination of the lungs confirmed a reduction in infiltration of B16F10 melanoma cells into the lung tissue.

FIG. 9 Quantification of the Anti-Metastatic Effects of Mab HUIV26 in the Chick Model. As described with regard to FIG. 8, B16F10 experimental metastasis was examined in the chick embryo model. The figure shows quantification of effects of Mab HUIV26 on B16F10 experimental metastasis. Data bars represent the mean number of tumor lesions per lung, per experimental condition+standard error. Experiments were completed 3 times with similar results. As described in Example III, to quantify the effects of Mab HUIV26 on B16F10 experimental metastasis, the number of B16F10 surface lesions were counted for each lung. As shown, in the presence of Mab HUIV26 (100 μg), the mean number of B16F10 lung foci was significantly (P<0.001) reduced by approximately 65% as compared to no treatment or control antibody while 1.0 μg of Mab HUIV26 per embryo exhibited little effect.

FIG. 10 Effect of Mab HUIV26 on Lesion Formation by B16F10 Melanoma Cell Injection in the Mouse Model. To evaluate whether Mab HUIV26 impacts tumor cell metastasis in a murine model, B16F10 experimental lung metastasis was examined. Mice were injected with either B16F10 cells in the presence or absence of Mab HUIV26 or an isotype matched control antibody (100 μg/mouse). Mice were treated intraperitoneally (100 μg/injection) for 7 days at which time they were sacrificed, lungs removed and the number of lung tumor lesions quantified. The figure shows representative examples of lungs from each experimental condition following injection of B16F10 melanoma cells. Experiments were completed 3 times with similar results. As described in Example IV, extensive B16F10 melanoma lesions could be detected on the surface of the murine lungs while a significant reduction in tumor lung lesions were observed on lungs from mice treated with Mab HUIV26.

FIG. 11 Quantification of the Anti-Metastatic Effects of Mab HUIV26 in the Mouse Model. To evaluate whether Mab HUIV26 impacts tumor cell metastasis in a murine model, B16F10 experimental lung metastasis was examined. Mice were injected with either B16F10 cells in the presence or absence of Mab HUIV26 or an isotype matched control antibody (100 μg/mouse). Mice were treated intraperitoneally (100 μg/injection) for 7 days at which time they were sacrificed, lungs removed and the number of lung tumor lesions quantified. The figure shows quantification of effects of Mab HUIV26 on B16F10 experimental metastasis. Arrows indicate examples of lung tumor lesions. Data bars represent the mean number of tumor lesions per lung, per experimental condition+standard error. Experiments were completed 3 times with similar results. As described in Example IV, the number of lung surface lesions were counted. Mab HUIV26 significantly (P<0.05) inhibited B16F10 experimental metastasis by approximately 50% as compared to either no treatment or treatment with an isotype matched control antibody.

FIG. 12 Effect of Mab HUIV26 on P21^{CIP1} mRNA Expression. To examine the effects that a function-blocking Mab directed to a cryptic collagen site has on the relative levels of CDK inhibitor P21^{CIP1}, real time quantitative RT-PCR analysis was performed. Non-tissue culture plates were coated (10.0 μg/ml) with denatured collagen type-IV. Tumor cells (B16F10 melanoma) were resuspended in the presence (100 μg/ml) or absence of Mab HUIV26 or an isotype matched control antibody and seeded onto the coated plates. Following a 12-hour incubation period, mRNA was prepared. The figure shows quantification of relative abundance of P21^{CIP1} mRNA within B16F10 tumor cells following treatment with Mab HUIV26 or an isotype matched control antibody. As described in Example V, the relative level of P21^{CIP1} mRNA was increased by approximately 2.3-fold when tumor cells (B16F10) were allowed to interact with denatured collagen type-IV in the presence or absence of Mab HUIV26 as compared to an isotype matched non-specific control antibody. Experiments were completed 2 to 3 times with similar results.

FIG. 13 Western Blot to Evaluate P21^{CIP1} Protein Levels. To examine the effects that a function-blocking Mab directed to cryptic collagen site has on the relative levels of CDK inhibitor P21^{CIP1}, Western blot analysis was performed. Non-tissue culture plates were coated (10.0 μg/ml) with denatured collagen type-IV. Tumor cells (B16F10 melanoma) were resuspended in the presence (100 μg/ml) or absence of Mab HUIV26 or an isotype matched control antibody and were seeded onto the coated plates. Following a 12-hour incubation period cell lysates were prepared. The figure shows Western blot analysis of relative abundance of P21^{CIP1} mRNA within B16F10 tumor cells following treatment with Mab HUIV26 or an isotype matched control antibody. As described in Example VI, incubation of B16F10 tumor cells plated on denatured collagen type-IV with Mab HUIV26 resulted in an approximately 2-fold increase in expression of P21^{CIP1} as compared to either no treatment or treatment with an isotype matched control non-specific antibody. Experiments were completed 2 to 3 times with similar results.

FIG. 14 Inhibition of αvβ3-Mediated Ligation of the HUIV26 Cryptic Collagen Epitope Increases TSP-1 Expression in Melanoma Cells. A. As described in Example VII, incubation of cells with Mab HUIV26, as compared to isotype-matched controls, resulted in an approximately 7-fold increase in the relative levels of TSP-1 mRNA. B. As also described in Example VII, incubation of cells with Mab LM609, as compared to isotype-matched controls, resulted in an approximately 8-fold increase in the relative levels of TSP-1 mRNA.

FIG. 15 Inhibition of αvβ3-Mediated Ligation of the HUIV26 Cryptic Collagen Epitope Increases IGFBP-4 expression. As described in Example VIII, incubation of cells with Mab HUIV26, as compared to isotype-matched controls, resulted in an approximately 115-fold increase in the relative levels of IGFBP-4 RNA.

FIG. 16 Inhibition of αvβ3-Mediated Ligation of the HUIV26 Cryptic Collagen Epitope Suppresses Id-1 Expression. As described in Example IX, incubating M21 cells in the presence of Mab HUIV26, as compared to isotype-matched control antibody treatment, resulted in a nearly 2-fold decrease in the relative levels of Id-1.

FIG. 17 Effect of Mab HUI77 on P21^{CIP1} mRNA Expression. To examine the effects that a function-blocking Mab directed to a cryptic collagen site has on the relative levels of CDK inhibitor P21^{CIP1}, real time quantitative RT-PCR analysis was performed. Non-tissue culture plates were coated (10.0 μg/ml) with denatured collagen type-IV. HUVECs were resuspended in the presence (100 μg/ml) or absence of Mab HUI77 or an isotype-matched control antibody and seeded onto the coated plates. Following a 12-hour incubation period, mRNA was prepared. The figure shows quantification of relative abundance of P21^{CIP1} mRNA within HUVECs following treatment with Mab HUI77 or an isotype matched control antibody. As described in Example XI, the relative level of P21^{CIP1} mRNA was increased significantly when HUVECs were allowed to interact with denatured collagen type-IV in the presence or absence of Mab HUI77 as compared to an isotype-matched non-specific control antibody.

FIG. 18 Effect of Mab HUI77 on P27^{KIP1} mRNA Expression. To examine the effects that a function-blocking Mab directed to a cryptic collagen site has on the relative levels of CDK inhibitor P27^{KIP1}, real time quantitative RT-PCR analysis was performed. Non-tissue culture plates were coated (10.0 μg/ml) with denatured collagen type-IV. HUVECs were resuspended in the presence (100 μg/ml) or absence of Mab HUI77 or an isotype-matched control antibody and seeded onto the coated plates. Following a 12-hour incubation period, mRNA was prepared. The figure shows quantification of relative abundance of P27^{KIP1} mRNA within HUVECs following treatment with Mab HUI77 or an isotype matched control antibody. As described in Example XII, the relative level of P27^{KIP1} mRNA was increased significantly when HUVECS were allowed to interact with denatured collagen type-IV in the presence or absence of Mab HUI77 as compared to an isotype-matched non-specific control antibody.

FIG. 19 Inhibition of αvβ3-Mediated Ligation of the HUIV26 Cryptic Collagen Epitope Increases TSP-1 Expression in HUVECs. As described in Example VII, incubation of cells with Mab HUIV26, as compared to isotype-matched controls, resulted in an approximately 6-fold increase in the relative levels of TSP-1 mRNA.

FIG. 20 Inhibition of αvβ3-Mediated Ligation of the HUIV26 Cryptic Collagen Epitope Increases IGFBP-4 Expression in HUVECs. As described in Example VIII, incubation of cells with Mab HUIV26, as compared to isotype-matched controls, resulted in a greater than 10-fold increase in the relative levels of IGFBP-4 mRNA.

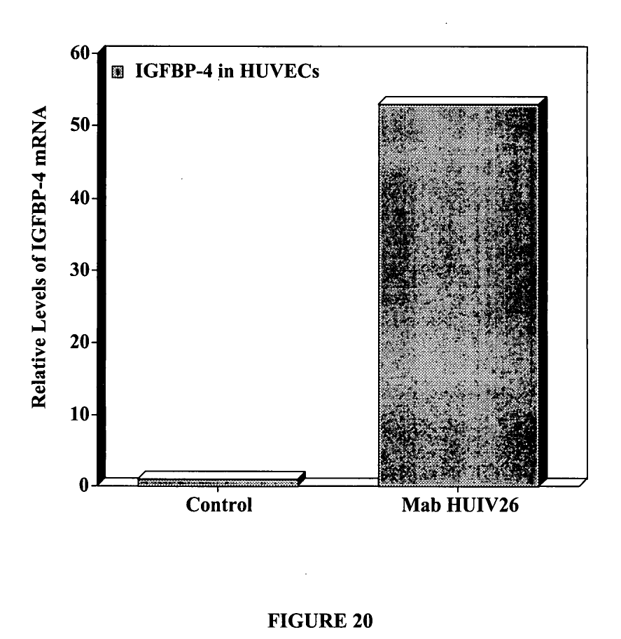

FIG. 21 Inhibition of Cellular Interactions by CLK-Peptide Enhances Expression of P27^{KIP1}. As described in Example XIII, Western Blot analysis of proteins from tumor cells incubated with CLK-peptide showed a significant upregulation of P27^{KIP1}.

FIG. 22 Inhibition of Cellular Interactions by CLK-Peptide Enhances Expression of P21^{CIP1}. As described in Example XIV, Western Blot analysis of proteins from tumor cells incubated with CLK-peptide showed a significant upregulation of P21^{CIP1}.

### DETAILED DESCRIPTION OF THE INVENTION

In describing the present invention, the following terms will be employed, and are intended to be defined as indicated below. Unless otherwise indicated, all terms used herein have the same ordinary meaning as they would to one skilled in the art of the present invention.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### Definitions

I. Antagonists of Cryptic Epitopes of ECM Components

Antagonists of cryptic epitopes of ECM components bind to cryptic epitopes of ECM components. As used herein, “antagonists” refers to molecules or compounds including, but not limited to, antibodies, peptides, polypeptides, cyclic peptides, oligonucleotides, and small molecule compounds. Methods for preparing and identifying candidate antagonists of cryptic epitopes of ECM components are described in, e.g., U.S. Ser. No. 09/478,977 (U.S. Pub. No. 2003/0113331); U.S. Publication No. 2004/0242490 A1; WO 2004/073649; U.S. Publication No. 2004/224896 A1, and; WO 2004/087734 (the disclosures of which are incorporated herein by reference in their entirety) as well as in the present application.

II. Cryptic Epitopes of ECM Components

As used herein, an “ECM component” is a component of the non-cellular compartment. ECM components include, e.g., collagen, fibrinogen, laminin, elastin, thrombospondin, tenascin, osteopontin, fibronectin and vitronectin, as well as other proteins and molecules found in association with these ECM components or found in the same location as these ECM components (Gustafsson, E., et al., R. Exp. Cell Res. 2000, 261: 52-68; Werb, Z., et al., Ann. N.Y. Acad. Sci. 1998, 857:110-118, and; Heissig, et al., Curr. Opin. Hematol. 2003, 10: 136-141).

The methods of the invention contemplate the use of antagonists that specifically bind to a cryptic epitope of an ECM component, including an ECM component from any animal. For example, collagens may be from any mammal such as rat, mouse, pig, rabbit, etc. or from a bird such as chicken. Generally, a collagen is an extracellular matrix protein containing a [Gly-Xaa-Xaa]ₙ sequence. Collagen types are well known in the art (see, e.g., Olsen, B. R., Curr. Op. Cell. Biol. 1995, 5:720-727; Kucharz, E. J. The Collagens: Biochemistry and Pathophysiology. Springer-Verlag, Berlin, 1992; Kunn, K. in Structure and Function of Collagen Types, eds. R. Mayne and R. E. Burgeson, Academic Press, Orlando; U.S. Publication No. 2003/0113331). Human collagens are preferred collagens. Denatured collagen refers to collagen that has been treated such that it no longer predominantly assumes the native triple helical form. Denaturation can be accomplished by heating the collagen. Collagen can be denatured by heating for about 15 minutes at about 100° C. Denaturation also can be accomplished by treating the collagen with a chaotropic agent. Suitable chaotropic agents include, for example, guanidinium salts. Denaturation of a collagen can be monitored, for example, by spectroscopic changes in optical properties such as absorbance, circular dichroism or fluorescence of the protein, by nuclear magnetic resonance, by Raman spectroscopy, or by any other suitable technique. Denatured collagen refers to denatured full-length collagens as well as to fragments of collagen. A fragment of collagen can be any collagen sequence shorter than a native collagen sequence. For fragments of collagen with substantial native structure, denaturation can be effected as for a native full-length collagen. Fragments also can be of a size such that they do not possess significant native structure or possess regions without significant native structure of the native triple helical form. Such fragments are denatured all or in part without requiring the use of heat or of a chaotropic agent. The term denatured collagen encompasses proteolyzed collagen. Proteolyzed collagen refers to a collagen that has been fragmented through the action of a proteolytic enzyme. In particular, proteolyzed collagen can be prepared by treating the collagen with a metalloproteinase, such as MMP-1, MMP-2 or MMP-9, or by treating the collagen with a cellular extract containing collagen degrading activity. Proteolyzed collagen can also be that which occurs naturally at sites of ECM remodeling in a tissue.

Laminins are a large family of extracellular matrix glycoproteins. Laminins have been shown to promote cell adhesion, cell growth, cell migration, cell differentiation, neurite growth, and to influence the metastatic behavior of tumor cells. Laminin, of which there are at least ten isoforms, is a major component of basement membranes and has been shown to mediate cell-matrix attachment, gene expression, tyrosine phosphorylation of cellular proteins, and branching morphogenesis (Streuli, et al., J. Cell Biol. 1993, 129:591-603; Malinda and Kleinman, Int. J. Biochem. Cell Biol. 1996, 28:957-1959; Timpl and Brown, Matrix Biol. 1994, 14:275-281; Tryggvason, Curr. Op. Cell Biol. 1993, 5:877-882; Stahl, et al., J. Cell Sci. 1997, 110:55-63). Laminin binds to type-IV collagen, heparin, gangliosides, and cell surface receptors and promotes the adhesion and growth of various epithelial and tumor cells as well as neurite outgrowth. Laminin is thought to mediate cell-matrix interactions and to be a structural component of all basement membranes binding to collagen type-IV, heparin sulfate proteoglycan, and nidogen-entactin. The laminin molecule is composed of three polypeptide chains (α, β, and γ) assembled into a cross-shaped structure. Different α, β, and γ chains may be combined, which accounts for the large size of the laminin family (Jones, J. C. R. et al., Micr. Res. Tech. 2000, 51:211-213; Patarroyo, M. et al., Semin. Cancer Biol. 2002, 12:197-207).

An epitope is that amino acid sequence or sequences that are recognized by an antagonist of the invention. An epitope can be a linear peptide sequence or can be composed of noncontiguous amino acid sequences. An antagonist can recognize one or more sequences, therefore an epitope can define more than one distinct amino acid sequence target. The epitopes recognized by an antagonist can be determined by peptide mapping and sequence analysis techniques well known to one of skill in the art.

A “cryptic epitope of an ECM component” is an epitope of an ECM component protein sequence that is not exposed for recognition within a native ECM component, but is capable of being recognized by an antagonist of a denatured or proteolyzed ECM component. Sequences that are not exposed, or are only partially exposed, in the native structure are potential cryptic epitopes. If an epitope is not exposed, or only partially exposed, then it is likely that it is buried within the interior of the molecule. The sequence of cryptic epitopes can be identified by determining the specificity of an antagonist. Candidate cryptic epitopes also can be identified, for example, by examining the three-dimensional structure of a native ECM component.

III. Angiogenesis and Diseases Potentially Treated by Inhibitors of Angiogenesis

As used herein, the terms “angiogenesis inhibitory,” “angiogenesis inhibiting” or “anti-angiogenic” include vasculogenesis, and are intended to mean effecting a decrease in the extent, amount, or rate of neovascularization. Effecting a decrease in the extent, amount, or rate of endothelial cell proliferation or migration in the tissue is a specific example of inhibiting angiogenesis.

The term “angiogenesis inhibitory composition” refers to a composition which inhibits angiogenesis-mediated processes such as endothelial cell migration, proliferation, tube formation and subsequently leading to the inhibition of the generation of new blood vessels from existing ones, and consequently affects angiogenesis-dependent conditions.

As used herein, the term “angiogenesis-dependent condition” is intended to mean a condition where the process of angiogenesis or vasculogenesis sustains or augments a pathological condition, or beneficially influences normal physiological processes. Therefore, treatment of an angiogenesis-dependent condition in which angiogenesis sustains a pathological condition could result in mitigation of disease, while treatment of an angiogenesis-dependent condition in which angiogenesis beneficially influences normal physiological processes could result in, e.g., enhancement of a normal process.

Angiogenesis is the formation of new blood vessels from pre-existing capillaries or post-capillary venules. Vasculogenesis results from the formation of new blood vessels arising from angioblasts which are endothelial cell precursors. Both processes result in new blood vessel formation and are included in the meaning of the term angiogenesis-dependent conditions. Similarly, the term “angiogenesis” as used herein is intended to include de novo formation of vessels such as those arising from vasculogenesis as well as those arising from branching and sprouting of existing vessels, capillaries and venules.

Examples of diseases in which angiogenesis plays a role in the maintenance or progression of the pathological state are listed herein in the Background of the Invention. Additional diseases are known to those skilled in the art and are similarly intended to be included within the meaning of “angiogenesis-dependent condition” and similar terms as used herein.

IV. Cancers, Tumors, and Tissues

The methods of the invention are contemplated for use in treatment of a tumor tissue of a patient with a tumor, solid tumor, a metastasis, a cancer, a melanoma, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include, but are not limited to, tumors of the skin, melanoma, lung, pancreas, breast, colon, laryngeal, ovarian, prostate, colorectal, head, neck, testicular, lymphoid, marrow, bone, sarcoma, renal, sweat gland, and the like tissues. Further examples of cancers treated are glioblastomas.

A tissue to be treated is a retinal tissue of a patient with diabetic retinopathy, macular degeneration or neovascular glaucoma and the angiogenesis to be inhibited is retinal tissue angiogenesis where there is neovascularization of retinal tissue.

Thus, methods which inhibit angiogenesis in a diseased tissue ameliorate symptoms of the disease and, depending upon the disease, can contribute to cure of the disease. In embodiments, the invention contemplates inhibition of angiogenesis in a tissue. The extent of angiogenesis in a tissue, and therefore the extent of inhibition achieved by the present methods, can be evaluated by a variety of methods, such as are described herein.

Any of a variety of tissues, or organs comprised of organized tissues, can support angiogenesis in disease conditions including skin, muscle, gut, connective tissue, joints, bones and the like tissue in which blood vessels can invade upon angiogenic stimuli. Thus, in one embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In the absence of neovascularization of tumor tissue, the tumor tissue does not obtain the required nutrients, slows in growth, ceases additional growth, regresses and ultimately becomes necrotic resulting in killing of the tumor. The present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because their formation requires vascularization of a primary tumor so that the metastatic cancer cells can exit the primary tumor and their establishment in a secondary site requires neovascularization to support growth of the metastases.

The invention also contemplates the practice of the method in conjunction with other therapies such as conventional chemotherapy directed against solid tumors and for control of establishment of metastases. The administration of an angiogenesis inhibitor is typically conducted during or after chemotherapy, although it is preferable to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

V. Patients

The invention contemplates treatment of patients including human patients. The term patient as used in the present application refers to all different types of mammals including humans and the present invention is effective with respect to all such mammals. The present invention is effective in treating any mammalian species which have a disease associated with angiogenesis or which reduction of angiogenesis would result in treatment of a condition including tumor metastasis, tumor growth, cell adhesion, cell proliferation or cell migration. The present invention has particular application to agricultural and domestic mammalian species.

### Modes of Carrying out the Invention

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

VI. Antagonists of Cryptic Epitopes of ECM Components

Potentially useful antagonists that specifically bind to cryptic epitopes of ECM components have been described in the literature. Antagonists of cryptic collagen epitopes are described in U.S. Publication No. 2003/0113331, U.S. Publication No. 2004/0242490 A1 and WO 2004/073649. Antagonists of cryptic laminin epitopes are disclosed in U.S. Publication No. 2004/224896 and WO 2004/087734.

Antagonists described in U.S. Publication No. 2003/0113331 bind to a denatured collagen or collagens, and are reported to bind with substantially reduced affinity to the native form of the collagen or collagens. Antagonists useful in the methods of the present invention can have an affinity to the native form of collagen, or another ECM component, of about 1.5-fold lower than that for the denatured collagen or denatured ECM component. Antagonists of the present invention are preferably specific for any one of the denatured collagens, e.g., types-I, II, III, IV, V, VI, VII, VIII, IX, X, and combinations thereof.

In U.S. Publication No. 2004/0242490 A1 and WO 2004/073649, the described antagonists reportedly have a binding affinity to denatured collagen type-IV that is substantially greater than the binding affinity of the antagonist to native collagen type-IV. A “substantially greater affinity” is defined therein as a binding affinity at least 1.5-fold greater for the target compound (denatured collagen) as compared to the standard compound (native collagen).

In U.S. Publication No. 2004/224896 and WO 2004/087734, peptide antagonists of denatured laminin are also described as having a binding affinity to denatured laminin that is “substantially greater” than the binding affinity of the antagonists to native laminin. “Substantially greater affinity” is defined therein as a binding affinity at least 1.5-fold greater for the target compound as compared to the standard compound and, more preferably, at least 10-fold greater and, most preferably, at least 100-fold greater. The selective antagonists are specific for denatured laminin (the target compound) and the binding affinities of the selective antagonists are compared to native laminin (the standard compound).

VI. A. Antibody Antagonists

Antagonists of the present invention include denatured ECM component antagonists in the form of antibodies which bind to a denatured ECM component or components but bind to a native ECM component or components with a substantially reduced affinity.

Antibodies useful in the invention can be monoclonal or polyclonal. In one embodiment, antibodies used are monoclonal. A monoclonal antibody of this invention comprises antibody molecules that immunoreact with a denatured ECM component, but immunoreact with a substantially reduced affinity with the native form of the ECM component.

Monoclonal antibodies which preferentially bind to denatured collagen include monoclonal antibodies having the immunoreaction characteristics of Mab HUIV26.

Antibody antagonists of the invention can be generated according to a number of methods known to one of skill in the art. For example, an animal can be immunized with a denatured collagen or fragment thereof. Antibodies thus generated can be selected both for their ability to bind to denatured or proteolyzed ECM components and for a substantially reduced affinity for the native form of the same ECM component. Antibodies can, for example, be generated by the method of “subtractive immunization” (see, e.g., Brooks, P. C. et al., J. Cell. Biol. 1993, 122:1351-1359 and U.S. Publication No. 2003/0113331).

The subtractive immunization technique allows one to experimentally manipulate the immune response within mice to selectively enhance an immune response to a rare and/or low abundant epitope within a mixture of common highly antigenic epitopes. As described in U.S. Publication No. 2003/0113331 with regard to preparation of antibodies that selectively bind to denatured collagen, the method can be carried out using an ECM component as follows: mice are injected intraperitoneally with a native ECM component. At 24 and 48 hours following the injections of the native ECM component, the mice are injected with the tolerizing agent, cyclophosphamide, to kill activated B-cells that would produce antibodies directed to common immunodominant epitopes within the native ECM component. Following the tolerization protocol, the mice are next injected with thermally denatured human ECM component to stimulate an immune response to epitopes exposed following thermal denaturation. The ECM component can be denatured, e.g., by boiling for 15 minutes or by proteolysis. The injections of the thermally denatured ECM component are given every three weeks for a total of 4 to 5 injections. Sera from each mouse is tested for immunoreactivity with both the native and denatured ECM components. The mice demonstrating the highest titer for reactivity to the denatured ECM component as compared to the native ECM component are used for the production of hybridomas. Spleen cells from the selected mice are fused with myeloma cells by standard techniques. Individual hybridoma clones are tested for the production of antibody to either native or denatured ECM component. Hybridoma clones are selected that produce antibodies that demonstrate a selective reactivity to the denatured ECM component as compared to the native ECM component. Mabs are purified by standard techniques.

As used in this application, the term “antibody” or “antibody molecule” refers to a population of a immunoglobulin molecules and/or immunological active portions of those particular immunoglobin molecules that contain the portion of an antibody which binds to its antigens, also known as the “antibody-combining site.”

The term “antibody” also includes molecules which have been engineered through the use of molecular biological technique to include only portions of the native molecule as long as those molecules have the ability to bind to a particular antigen with the required specification. Such alternative antibody molecules include classically known portions of the antibodies molecules and single chain antibodies.

Antibodies for use in the present invention are intact immunoglobulin molecules, substantially intact immunoglobulin molecules and those portions of an immunoglobulin molecule that contain the paratope, including those portions known in the art as Fab, Fab′, F(ab′)₂ and F(v), also referred to as antibody fragments.

The invention embodies a truncated immunoglobulin molecule comprising a Fab fragment derived from a monoclonal antibody of this invention. The Fab fragment, lacking Fc receptor, is soluble, and affords therapeutic advantages in serum half life and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by proteolysis using papain and pepsin, respectively, on substantially intact antibodies by methods that are well known. See for example, U.S. Pat. No. 4,342,566 to Theofilopolous and Dixon. Fab′ antibody portions also are well known and are produced from F(ab′)₂ portions, followed by reduction of disulfide bonds linking the two heavy chains as with mercaptoethanol, and followed by alkylation of the resulting protein mercaptan with a reagent such as iodoacetamide.

The term monoclonal antibody as used herein refers to an antibody molecule population that has only one particular antibody combining site and is capable of immunoreacting with a particular epitope. A monoclonal antibody typically displays a single binding affinity for that epitope and such binding can be measured by standard amino acids. Monoclonal antibodies that are useful in this invention may also contain a number of different antibody combining sites wherein each antibody combining site is specific for a particular epitope. Examples of such monoclonal antibodies include biospecific monoclonal antibodies. Monoclonal antibodies contemplated by the present invention also include monoclonal antibodies that are produced by various methods including traditional monoclonal antibodies technology and modern molecular techniques which isolate the antibody combining site of a particular antibody and express it as either a part of a immunological molecule or as part of another molecule.

A monoclonal antibody can be composed of antibodies produced by clones of a single cell called a hybridoma that produces only one kind of antibody molecule. The hybridoma cell is formed by fusing an antibody-producing cell and a myeloma or other self-perpetuating cell line. The preparation of such antibodies was first described by Kohler and Milstein, Nature 1975, 256:495-497. Additional methods are described by Zola, Monoclonal Antibodies: A Manual of Techniques, CRC Press, Inc. (1987).

A monoclonal hybridoma culture is initiated comprising a nutrient medium containing a hybridoma that secretes antibody molecules of the appropriate specificity. The culture is maintained under conditions and for a time period sufficient for the hybridoma to secrete the antibody molecules into the medium. The hybridoma supernatant so prepared can be screened for the presence of antibody molecules that immunoreact with cryptic epitopes of ECM components.

To form the hybridoma from which the monoclonal antibody is produced, a myeloma or other self-perpetuating cell line is fused with lymphocytes obtained from the spleen of a mammal hyperimmunized with a source of a cryptic epitope of an ECM component.

It is preferred that the myeloma cell line used to prepare a hybridoma be from the same species as the lymphocytes. A mouse of the strain 129 G1X⁺ is typically the preferred mammal. Suitable mouse myelomas for use in the present invention include the hypoxanthine-aminopterin-thymidine-sensitive (HAT) cell lines P3×63-Ag8.653, and Sp2/0-Ag14 that are available from the American Type Culture Collection, Rockville, Md., under the designations CRL 1580 and CRL 1581, respectively.

Splenocytes are typically fused with myeloma cells using a space inhibitor such as polyethylene glycol (PEG) 1500. Fused hybrids are selected by their sensitivity to a selective growth medium, such as HAT (hypoxanthine aminopterin thymidine) medium. Hybridomas producing a monoclonal antibody of this invention can be identified using the enzyme linked immunosorbent assay (ELISA).

Media useful for the preparation of these compositions are both well known in the art and commercially available and include synthetic culture media, media derived from inbred mice and the like. An exemplary synthetic medium is Dulbecco's minimal essential medium (DMEM; Dulbecco et al., Virol. 1959, 8:396, 1959) supplemented with 4.5 g/L glucose, 20 nM glutamine, and 20% fetal calf serum. An exemplary inbred mouse strain is the Balb/c.

Alternatively, the monoclonal antibody may be produced using cloning methods to isolate the gene(s) encoding the monoclonal antibody. Such techniques are well known in the art. See, for example, the method of isolating monoclonal antibodies from an immunological repertoire as described by Sastry et al., Proc. Natl. Acad. Sci. USA 1989, 86:5728-5732; and Huse et al., Science 1989, 246:1275-1281.

Antibodies, whether polyclonal or monoclonal, can be raised against the desired proteins or peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

Humanized monoclonal antibodies offer advantages over murine monoclonal antibodies, particularly insofar as they can be used therapeutically in humans. Human antibodies are not cleared from the circulation as rapidly as “foreign” antigens, and do not activate the immune system in the same manner as foreign antigens and foreign antibodies. Methods of preparing “humanized” antibodies are known in the art, and can be applied to the antibodies of the present invention.

Thus, the invention contemplates, in one embodiment, a monoclonal antibody of this invention that is humanized by grafting to introduce components of the human immune system without substantially interfering with the ability of the antibody to bind antigen.

The antibody of the invention can also be a fully human antibody such as those generated, for example, by selection from an antibody phage display library displaying human single chain or double chain antibodies such as those described in de Haard, H. J. et al., J. Biol. Chem. 1999, 274:18218-30 and in Winter, G. et al., Annu. Rev. Immunol. 1994, 12:433-55.

Humanized antibodies to denatured collagen are described in U.S. Ser. No. 09/995,529 (U.S. Publication No. 2003/0099655), the disclosure of which is hereby incorporated by reference in its entirety.

VI. B. Peptide and Polypeptide Antagonists

Preparation of Peptide and Polypeptide Antagonists

Peptides can be linear or cyclic, although particularly preferred peptides are cyclic. Longer polypeptides, e.g., of greater than about 100 residues, can be provided in the form of a fusion protein or protein fragment. Antagonists of native or denatured ECM components also can be polypeptides or peptides. The term polypeptide refers to a sequence of 3 or more amino acids connected to one another by peptide bonds between the alpha-amino group and carboxy group of contiguous amino acid residues. The term peptide as used herein refers to a series of two or more amino acid residues connected to one to the other as in a polypeptide.

It should be understood that a subject polypeptide need not be identical to the amino acid residue sequence of a cryptic epitope of an ECM component.

A subject polypeptide includes any analog, fragment or chemical derivative of a polypeptide antagonist of a cryptic epitope of an ECM component. Therefore, a present polypeptide can be subject to various changes, substitutions, insertions, and deletions where such changes provide for certain advantages in its use. In this regard, an antagonist polypeptide of this invention corresponds to, rather than is identical to, the sequence of a recited peptide where one or more changes are made and it retains the ability to function as an antagonist in one or more of the assays as defined herein.

The polypeptides or peptides of the present invention may be a peptides or polypeptides derivative that include those residue or chemical changes including amides, conjugates with proteins, cyclic peptides, polymerized peptides and analogs of fragments of chemically modified peptides or proteins and other types of derivatives.

The term “analog” includes any polypeptide having an amino acid residue sequence substantially identical to a given sequence. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” as used in this application includes chemically derivatized residues that are used to replace a non-derivatized residue in a peptide or polypeptide that results in a peptide or polypeptide that maintains the desired function.

Polypeptide antagonists of the present invention can have sequences in which one or more conservative or non-conservative substitutions have been made, usually up to about 30 (number) percent. Up to about 10 (number) percent of the amino acid residues can be substituted. Additional residues may also be added at either terminus of a polypeptide for the purpose of providing a “linker” by which the polypeptides of this invention can be conveniently affixed to a label or solid matrix, or carrier.

The term “chemical derivative” as used in this application refers to polypeptide or peptide having amino acid sequence resitives that are changed or derivatized chemically by using a reaction with a functional side group. Other contemplated derivitizations of peptides or polypeptides includes a chemical derivative which uses backbone modifications including α-amino acids substitutions, such as N-methyl, N-ethyl, N-propyl and other similar substitutions to replace various residues within the backbone. Other potential derivatives utilizing backbone modifications include α-carbonyl substitutions such as thioester, thioamide, guanidino, and other similar substitutions. The present invention also contemplates the use of derivitized molecules which include pre-amino acid groups which have been derivitized to form hydroclorides, p-toleune sulfonyl groups carbobenzoxy groups, t-butyloxycarbonyl groups, chloroacetyl groups or formyl groups. The free carboxyl groups typically may be derivitized to form salts, methyl and ethyl esters or other types of esters or hydrazides. In the free hydroxyl groups may be derivatized to form O-acyl or O-alkyl derivatives of those peptides or polypeptides.

Labels, solid matrices and carriers that can be used with the polypeptides of this invention are described herein below.

Amino acid residue linkers are usually at least one residue and can be 40 or more residues, more often 1 to 10 residues, but do not form a cryptic epitope of an ECM component. Typical amino acid residues used for linking are tyrosine, cysteine, lysine, glutamic and aspartic acid, or the like. In addition, a subject polypeptide can differ, unless otherwise specified, from the natural sequence of the ECM cryptic epitope ligand by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong the half-life of the polypeptides in solutions, particularly biological fluids where proteases may be present. In this regard, polypeptide cyclization is also a useful terminal modification because of the stable structures formed by cyclization and in view of the biological activities observed for such cyclic peptides.

Any peptide of the present invention may be used in the form of a pharmaceutically acceptable salt. Suitable acids which are capable of forming salts with the peptides of the present invention include inorganic acids such as trifluoroacetic acid (TFA) hydrochloric acid (HC), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HC and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine) and optionally substituted ethanolamines (e.g. ethanolamine and diethanolamine).

In addition, a peptide useful in the methods of this invention can be prepared without including a free ionic salt in which the charged acid or base groups present in the amino acid residue side groups (e.g., Arg, Asp, and the like) associate and neutralize each other to form an “inner salt” compound.

A peptide of the present invention can be synthesized by any of the techniques that are known to those skilled in the polypeptide art, including recombinant DNA techniques. Synthetic chemistry techniques, such as a solid-phase Merrifield-type synthesis can be advantageous for reasons of purity, antigenic specificity, freedom from undesired side products, ease of production and the like. Summaries of the many techniques available can be found in, e.g., Steward et al., “Solid Phase Peptide Synthesis,” W. H. Freeman Co., San Francisco, 1969; Bodanszky, et al., “Peptide Synthesis,” John Wiley & Sons, Second Edition, 1976; J. Meienhofer, “Hormonal Proteins and Peptides,” Vol. 2, p. 46, Academic Press (New York), 1983; Merrifield, Adv. Enzymol. 1969, 32:221-96; Fields et al., Int. J. Peptide Protein Res. 1990, 35:161-214; U.S. Pat. No. 4,244,946 for solid phase peptide synthesis, and Schroder et al., “The Peptides,” Vol. 1, Academic Press (New York), 1965 (for classical solution synthesis). Appropriate protective groups usable in such synthesis are also described in J. F. W. McOmie, “Protective Groups in Organic Chemistry,” Plenum Press, New York, 1973.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

In solid phase synthesis, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

Linear polypeptides may be reacted to form their corresponding cyclic peptides. A method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol, sodium hydroxide solution is added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Alternative methods for cyclic peptide synthesis are described by Gurrath et al., Eur. J. Biochem., 210:911-921 (1992).

In addition, the antagonist can be provided in the form of a fusion protein. Fusion proteins are proteins produced by recombinant DNA methods known and described in the art, in which the subject polypeptide is expressed as a fusion with a second carrier protein such as a glutathione sulfhydryl transferase (GST) or other well-known carrier.

Thus, a polypeptide can be present in any of a variety of forms of peptide derivatives, including amides, conjugates with proteins, cyclized peptides, polymerized peptides, analogs, fragments, chemically modified peptides, and like derivatives.

Identification of Peptide and Polypeptide Antagonists

The invention contemplates use of denatured ECM component antagonists in the form of polypeptides. A polypeptide antagonist of a denatured ECM component can be any peptide or polypeptide capable of binding to a denatured ECM component, but one that binds to the native form of the ECM component with substantially reduced affinity.

Examples of peptide antagonists of denatured collagen are described in U.S. Publication No. 2004/0242490 A1 and WO 2004/073649, “CLK-Peptide and SLK-Peptide.” One preferred denatured collagen type-IV selective peptide antagonist contemplated for use in the present invention is the CLK-peptide, described in the aforementioned publications. CLK-peptide binds to denatured collagen type-IV with high specificity. The amino acid sequence of CLK peptide is NH₂—C-L-K-Q-N-G-G-N—F—S-L-G-COOH (SEQ ID NO: 15). The CLK-peptide binds to regions within denatured collagen type-IV and inhibits cellular interactions with denatured collagen type-IV.

Another selective denatured collagen type-IV peptide antagonist contemplated for use in the present invention is SLK-peptide. SLK-peptide binds with high specificity to denatured collagen type-IV and inhibits cellular interactions with denatured collagen type-IV. The amino acid sequence of SLK-peptide is NH₂—S-L-K-Q-N-G-G-N—F—S-L-C—COOH (SEQ ID NO: 16).

A further preferred selective denatured collagen type-IV peptide antagonist contemplated for use in the present invention is KGGCLK peptide (SEQ ID NO: 13). KGGCLK peptide (SEQ ID NO: 13) binds with high specificity to denatured collagen type-IV and inhibits cellular interactions with denatured collagen type-IV. The amino acid sequence of KGGCLK peptide is NH₂—K-G-G-C-L-K-Q-N-G-G-N—F—S-L-G-G-K—COOH (SEQ ID NO: 17).

Peptide antagonists of denatured laminin have been disclosed in U.S. Publication No. 2004/224896 and WO 2004/087734. One denatured laminin antagonist described in these publications having the amino acid sequence NH₂—S-T-Q-N-A-S-L-L-S-L-T-V—C—COOH (SEQ ID NO: 14). Another preferred denatured laminin selective antagonist for use in the present invention is a peptide having the amino acid sequence NH₂—K-G-G-C—S-T-Q-N-A-Q-L-L-S-L-I—V-G-K-A-COOH (STQ-peptide; SEQ ID NO: 18). Another preferred denatured laminin selective antagonist for use in the present invention is a peptide having the amino acid sequence NH₂—K-G-G-S-T-Q-N-A-Q-L-L-S-L-I—V-G-K-A-COOH (STQ-peptide-S; SEQ ID NO: 19).

The identification of denatured ECM component antagonist peptides having selectivity for denatured ECM components can readily be identified in a typical inhibition of binding assay, such as the ELISA assay.

Peptide and polypeptide antagonists of denatured ECM components can be generated by a number of techniques known to one of skill in the art. For example, a two-hybrid system (e.g., Fields, S., Nature 1989, 340:245-6) can use a fragment of an ECM component, e.g., collagen or laminin, as “bait” for selecting protein antagonists from a library that bind to the fragment. The library of potential antagonists can be derived from a cDNA library, for example. The potential antagonists can also be variants of known ECM component binding proteins. Such proteins can be randomly mutagenized or subjected to gene shuffling, or other available techniques for generating sequence diversity.

Peptide and polypeptide antagonists also can be identified by techniques of molecular evolution. Libraries of proteins can be generated by mutagenesis, gene shuffling or other available techniques for generating molecular diversity. Protein pools representing numerous variants can be selected for their ability to bind to denatured ECM components, for instance by passing such protein pools over a solid matrix to which a denatured ECM component, e.g., denatured collagen, has been attached. Elution with gradients of salt, for example, can provide purification of variants with affinity for the denatured ECM component. A negative selection step also can be included whereby such pools are passed over a solid matrix to which a native ECM component has been attached. The filtrate will contain those variants within the pool that have a reduced affinity for the native form of the collagen. This method can be applied to the identification of antagonists having specificity for the denatured forms of other ECM components.

Peptide and polypeptide antagonists of the invention also can be generated by phage display. A randomized peptide or protein can be expressed on the surface of a phagemid particle as a fusion with a phage coat protein. Techniques of monovalent phage display are widely available (see, e.g., Lowman H. B. et al., Biochemistry 1991, 30:10832-8.) Phage expressing randomized peptide or protein libraries can be panned with a solid matrix to which a native ECM component molecule has been attached. Remaining phage do not bind the native molecule, or bind native molecules with substantially reduced affinity. The phages are then panned against a solid matrix to which the denatured ECM component has been attached. Bound phage are isolated and separated from the solid matrix by either a change in solution conditions or, for a suitably designed construct, by proteolytic cleavage of a linker region connecting the phage coat protein with the randomized peptide or protein library. The isolated phage can be sequenced to determine the identity of the selected antagonist.

In another embodiment, a polypeptide includes any analog, fragment or chemical derivative of a given polypeptide so long as the polypeptide is an antagonist of a denatured ECM component. Therefore, a present polypeptide can be subject to various changes, substitutions, insertions, and deletions where such changes provide for certain advantages in its use. In this regard, an antagonist polypeptide of this invention corresponds to, rather than is identical to, the sequence of a recited peptide where one or more changes are made and it retains the ability to function as a denatured ECM component antagonist.

VI. C. Other Antagonists of Cryptic Epitopes of ECM Components

Antagonists of the invention also can be small organic molecules, such as those natural products, or those compounds synthesized by conventional organic synthesis or combinatorial organic synthesis. Compounds can be tested for their ability to bind to a denatured ECM component for example by using the affinity-purification technique described herein.

Compounds also are selected for reduced affinity for the native form of the ECM component by a similar affinity-purification technique.

Antagonists of the invention also can be non-peptidic compounds, including, for example, oligonucleotides. Oligonucleotides, as used herein, refers to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g. phosphorothioate oligonucleotides). Oligonucleotides may present commonly found purine and pyrimidine bases such as adenine, thymine, guanine, cytidine and uridine, as well as bases modified within the heterocyclic ring portion (e.g., 7-deazaguanine) or in exocyclic positions. “Oligonucleotide” also encompasses heteropolymers with distinct structures that also present aromatic bases, including polyamide nucleic acids and the like.

An oligonucleotide antagonist of the invention can be generated by a number of methods known to one of skill in the art. In one embodiment, a pool of oligonucleotides is generated containing a large number of sequences. Pools can be generated, for example, by solid phase synthesis using mixtures of monomers at an elongation step. The pool of oligonucleotides is sorted by passing a solution containing the pool over a solid matrix to which a denatured ECM component or fragment thereof has been affixed. Sequences within the pool that bind to the denatured ECM component are retained on the solid matrix. These sequences are eluted with a solution of different salt concentration or pH. Sequences selected are subjected to a second selection step. The selected pool is passed over a second solid matrix to which the native ECM component has been affixed. The column retains those sequences that bind to the native ECM component, thus enriching the pool for sequences specific for the denatured ECM component. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

VII. Identification of Antagonists of Cryptic Epitopes of ECM Components

Potentially useful antagonists of cryptic epitopes of ECM components have been described in U.S. Publication No. 2003/0113331; U.S. Publication No. 2004/0242490 A1; WO 2004/073649; U.S. Publication No. 2004/224896 A1, and; WO 2004/087734. In the identification methods of the invention, candidate antagonists are evaluated for their ability to bind to denatured ECM components, and furthermore can be evaluated for their potency in altering metastasis, angiogenesis, and other tumor development processes, in a tissue. Measurement of binding of antagonists to denatured or native ECM components in the solid phase can be accomplished, e.g., using an enzyme-linked-immunosorbent assay (ELISA), described in these publications and herein. The ELISA is commonly used and well-known to those of skill in the art.

The ELISA also can be used to identify compounds which exhibit increased specificity for denatured, as compared to the native forms of ECM components. The specificity assay is conducted by running parallel ELISAs in which a potential antagonist is screened concurrently in separate assay chambers for the ability to bind denatured and native ECM components. Another technique for measuring apparent binding affinity familiar to those of skill in the art is a surface plasmon resonance technique (analyzed on a BIACORE 2000 system) (Liljeblad, et al., Glyco. J. 2000, 17: 323-329). Standard measurements and traditional binding assays are described by Heeley, R. P., Endocr. Res. 2002, 28: 217-229.

Antagonists of denatured ECM components can also be identified by their ability to compete for binding with antagonists useful in the present invention. For example, putative antagonists can be screened by monitoring their effect on the affinity of a known antagonist, such as antibody HUIV26, described in U.S. Publication No. 2003/0113331. Such antagonists likely have the same specificity as, and recognize the same cryptic epitope, as the antibodies themselves. Putative antagonists selected by such a screening method can bind either to the ECM component or to the antagonist. Antagonists can be selected from the putative antagonists by conventional binding assays to determine those that bind to the cryptic epitope of the ECM component but not to the known antagonist.

Antagonists can be identified by their ability to bind to a solid matrix containing a denatured ECM component. Such putative antagonists are collected after altering solution conditions, such as salt concentration, pH, temperature, etc. The putative antagonists are further identified by their ability to pass through, under appropriate solution conditions, a solid matrix to which a native ECM component has been affixed.

Antagonists useful in the invention can be assayed for their ability to influence tumor development processes, e.g., angiogenesis, tumor metastasis, cell adhesion, cell migration, and tumor growth in a tissue as well as their effect on angiogenesis-dependent conditions. Any suitable assay known to one of skill in the art can be used to monitor such effects. Several such assays are described herein.

VIII. Methods for Identifying Genes Modulated by Binding of an Antagonist to a Cryptic Epitope of an ECM Component

In methods of the invention, expression of at least one gene or protein is modulated by binding of an antagonist to a cryptic epitope of an ECM component. Methods for identifying modulated genes and proteins of the invention are provided in the examples.

Generally, cells that have been associated with a cryptic epitope of an ECM component are treated with the antagonist. Association of the cryptic epitope of the ECM component and the cells can be accomplished by various means. For example, as described in Example V, dishes can be coated with the cryptic epitope (in this example, denatured collagen type-IV was used) and the cells added to the coated dishes. The cryptic epitope can also be mixed or contacted with the cells in solution or media. After antagonist treatment, a comparison of gene expression or protein levels observed in either treated cells or untreated cells is then made. A panel of genes or proteins, or just one gene or protein, can be compared by these methods. Based on analyses of the gene expression or protein levels, modulated genes or proteins can be identified.

As used herein, the term “modulated” is intended to mean either upregulated or downregulated. Modulation of gene expression can be determined by quantitating nucleic acid, e.g., RNA or cDNA, from specific genes. In embodiments, the expression of a gene or protein is upregulated or downregulated at least 1.5-fold, relative to the control gene expression.

For example, as described in Example VIII, when M21 cells are allowed to interact with denatured collagen type-IV in the presence or absence of Mab HUIV26, which binds specifically to the cryptic collagen epitope HUIV26, IGFBP-4 RNA expression, as measured by Real Time Quantitative RT-PCR, increases 115 times relative to RNA expression measured when cells are treated with an isotype-matched control antibody. Incubation of cells with Mab HUIV26 resulted in an approximately 7-fold increase in the relative levels of TSP-1 RNA as compared to RNA expression by cells treated with isotype-matched controls.

Furthermore, as described herein, incubating M21 cells in the presence of Mab HUIV26 resulted in a nearly 2-fold decrease in the relative levels of Id-1 as compared to isotype-matched control antibody treatment.

Modulation of gene expression levels, including the levels of IGFBP-4, TSP-1, Id-1, and p21^{CIP1}, can be measured using methods well-known to those of skill in the art, e.g., Real Time quantitative RT-PCR. Primer sequences useful for detecting IGFBP-4, TSP-1, Id-1, and p21^{CIP1}, are given below in the Examples, and additional primer sequences for these genes as well as primer sequences for other genes identified as modulated in the methods of the invention can be determined by sequence analysis methods and using software as known and commonly used by those of skill in the art.

In addition to PCR techniques, other methods for detecting and quantifying nucleic acids are well known to those skilled in the art and have been described in the literature, including hybridization methods, e.g., Southern blotting, Northern blotting, etc., with subsequent quantification by known methods. Amplification techniques, including PCR, can be used prior to analysis using one of the above methods.

Expression of test genes can be compared to expression of an internal control gene, e.g., β2-Macroglobulin. Other suitable endogenous internal control genes and methods for identifying control genes in different tissues are well known to those of skill in the art. For example, methods for identifying control genes have been described by Vandesompele, et al., “Accurate normalization of real-time quantitative RT-PCR data by geometric averaging of multiple internal control genes,” Genome Biol. 2002, 3(7): research0034.1-research0034.11.

IX. Methods for Identifying Proteins Modulated by Antagonists to a Cryptic Epitope of an ECM Component

Modulation of protein levels, including TSP-1, IGFBP-4, Id-1, and P21^{CIP}, can be measured using methods described in the literature and well-known to those of skill in the art. Enzyme Linked Immunosorbent Assay (ELISA), Western Blot analysis, radioimmunoassay and immunoprecipitation are examples of methods that can be used to detect and quantitate the proteins of interest. Enzymatic assays, also well known in the art, can also be used where appropriate.

For example, as determined by Western Blotting and described herein, incubation of B16F10 tumor cells plated on denatured collagen type-IV with Mab HUIV26 resulted in an approximately 2-fold increase in expression of P21^{CIP1} as compared to either no treatment or treatment with an isotype-matched control non-specific antibody. No change in the relative levels of the control protein, actin, was observed under the different experimental conditions.

X. Gene Products and Proteins

Gene products or proteins identified and administered according to the methods of the invention include TSP-1, IGFBP-4, Id-1, and P21^{CIP}. Also contemplated for administration are polypeptide portions of IGFBP-4, wherein the portion of the gene product is an active portion having angiogenesis, metastasis or tumor development-inhibiting properties, or it has the ability to exert a beneficial effect on angiogenesis-dependent conditions. IGFBP-4 has been shown to be proteolyzed (see, e.g., Overgaard, J. Biol. Chem. 2000, 275(40):31128-33). It has been reported in the literature that a number of proteins that inhibit angiogenesis, including angiostatin, endostatin, pexstatin, tumstatin, laminin, and fibronectin, have increased anti-angiogenic activity when present in cleaved forms as compared to full-length forms. The resulting cleavage products possess anti-angiogenic activity. For example, the angiogenesis inhibitor, angiostatin, is derived from plasminogen, and the prothrombin kringle-2 domain is a cleavage product of prothrombin (Lee, et al., J. Biol. Chem. 1998, 273 (44):28805-12; Soff, G. A., Cancer Metastasis Rev. 2000, 19(1-2):97-107). A short peptide from matrix metalloproteinase-2 (MMP-2) has also been found to inhibit angiogenesis and tumor growth (U.S. Pub. No. 2002/0182215 A1, incorporated herein by reference in its entirety). Therefore, identified polypeptides, as well as naturally-occurring cleavage products, are contemplated for use according to the methods of the invention. The use of cryptic regions of ECM components having anti-angiogenic function are discussed in, e.g., Schenk, S., et al., Trends in Cell Biol. 2003, 13: 366-375 and Kalluri, R. Nat. Rev. Cancer 2003, 3: 422-433.

Gene products can be expressed from genes identified according to the methods of the invention by numerous methods known to those of skill in the art and described in the literature.

For example, recombinantly-produced proteins of the present invention can be directly expressed or expressed as fusion proteins. The recombinant protein can be purified by a combination of cell lysis (e.g., sonication, French press) and affinity chromatography. For fusion products, subsequent digestion of the fusion protein with an appropriate proteolytic enzyme can release the desired recombinant protein.

Polynucleotides containing genes identified using the methods of the present invention may be cloned, using standard cloning and screening techniques, from a cDNA library, (see for instance, Sambrook et al., Molecular Cloning: A Laboratory Manual, 2nd Ed., Cold Spring Harbor Laboratory Press, Cold Spring Harbor, N.Y. (1989)). These polynucleotides can also be obtained from natural sources such as genomic DNA libraries or can be synthesized using well known and commercially available techniques.

When genes of the present invention are used for the recombinant production of gene products or proteins of the present invention, the polynucleotide including the gene sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. Polynucleotides can also contain non-coding 5′ and 3′ sequences, such as transcribed, non-translated sequences, splicing and polyadenylation signals, ribosome binding sites and sequences that stabilize mRNA.

There are a number of methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Modifications of the technique, exemplified by the Marathon technology (Clontech Laboratories Inc.) for example, have significantly simplified the search for longer cDNAs. In the Marathon technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene-specific and adaptor-specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al., 1989. Preferred methods of introducing polynucleotides into host cells include, for instance, calcium phosphate transfection, DEAE-dextran mediated transfection, transfection, micro-injection, cationic lipid-mediated transfection, electroporation, transduction, scrape loading, ballistic introduction or infection.

Representative examples of appropriate hosts include, e.g., bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

As understood in the art, a great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., 1989. Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

The proteins of this invention, recombinant or synthetic, can be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). The protein may then be isolated from cells expressing the protein and further purified by standard protein chemistry techniques.

XI. Methods of Assaying Tumor Metastasis

Tumor metastasis can be measured by a number of techniques known to those of skill in the art and published in the literature. The Examples describe assaying tumor metastasis using the chick embryo model (Brooks et al., Meth. Mol. Biol 1999, 129:257-269; Testa et al., Cancer Res 1999, 59: 3812-3820), and the murine model (Vantyghem, et al., Cancer Res 2003, 63:4763-4765). Subsequent histological and immunofluorescence analyses can be performed as described in the literature (Brooks, et al., Cell 1996, 85:683-693; Brooks, et al., Science 1994, 264:569-571) and herein.

XII. Methods of Assaying Angiogenesis

Methods of measuring alterations in angiogenesis are well known in the art. For example, angiogenesis can be measured in the chick chorioallantoic membrane (CAM). This assay is referred to as the CAM assay. The CAM assay has been described in detail, and further has been used to measure both angiogenesis and neovascularization of tumor tissues. See Ausprunk et al., Am. J. Pathol., 1975, 79:597-618 and Ossonski et al., Cancer Res., 1980, 40:2300-2309. The CAM assay is a well-recognized assay model for in vivo angiogenesis because neovascularization of whole tissue is occurring, and actual chick embryo blood vessels are growing into the CAM or into the tissue grown on the CAM.

The CAM assay is particularly useful because there is an internal control for toxicity in the assay system. The chick embryo is exposed to any test reagent, and therefore the health of the embryo is an indication of toxicity.

Alterations in angiogenesis can also be measured using the in vivo rabbit eye model, referred to as the rabbit eye assay. The rabbit eye assay has been described in detail by others, and further has been used to measure both angiogenesis and neovascularization in the presence of angiogenic inhibitors such as thalidomide. See D'Amato et al., Proc. Natl. Acad. Sci. 1994, 91:4082-4085.

The rabbit eye assay is a well recognized assay model for in vivo angiogenesis because the neovascularization process, exemplified by rabbit blood vessels growing from the rim of the cornea into the cornea, is easily visualized through the naturally transparent cornea of the eye. Additionally, both the extent and the amount of stimulation or inhibition of neovascularization or regression of neovascularization can easily be monitored over time. Finally, the rabbit is exposed to any test reagent, and therefore the health of the rabbit is an indication of toxicity of the test reagent.

Another assay measures angiogenesis in the chimeric mouse:human mouse model and is referred to as the chimeric mouse assay. This assay is described herein, and in detail by others, as a method of measuring angiogenesis, neovascularization, and regression of tumor tissues. See Yan, et al. J. Clin. Invest. 1993, 91:986-996.

The chimeric mouse assay is a useful assay model for in vivo angiogenesis because the transplanted skin grafts closely resemble normal human skin histologically, and neovascularization of whole tissue is occurring wherein actual human blood vessels are growing from the grafted human skin into the human tumor tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of regression of new vessel growth. Furthermore, it is easy to monitor effects on the growth of any tissue transplanted upon the grafted skin, such as a tumor tissue. Finally, the assay is useful because there is an internal control for toxicity in the assay system. The chimeric mouse is exposed to any test reagent, and therefore the health of the mouse is an indication of toxicity.

To confirm the effects of a compound, e.g., IGFBP-4, on angiogenesis, the mouse Matrigel plug angiogenesis assay can be used. Various growth factors (IGF-1, bFGF or VEGF) (250 ng) and Heparin (0.0025 units per/ml) are mixed with growth factor reduced Matrigel as previously described (Montesano, et al., J. Cell Biol. 1983, 97: 1648-1652; Stefansson, et al., J. Biol. Chem. 2000, 276: 8135-8141). IGFBP-4 or control BSA (10 to 500 ng) can be included in the Matrigel preparations. In control experiments, Matrigel is prepared in the absence of growth factors. Mice are injected subcutaneously with 0.5 ml of the Matrigel preparation and allowed to incubate for one week. Following the incubation period, the mice are sacrificed and the polymerized Matrigel plugs surgically removed. Angiogenesis within the Matrigel plugs is quantified by two established methods, including immunohistochemical analysis and hemoglobin content (Furstenberger, et al., Lancet. 2002, 3: 298-302; Volpert, et al., Cancer Cell 2002, 2(6): 473-83.; Su, et al., Cancer Res. 2003, 63: 3585-3592). For immunohistochemical analysis, the Matrigel plugs are embedded in OCT, snap frozen and 4 μm sections prepared. Frozen sections are fixed in methanol/acetone (1:1). Frozen sections are stained with polyclonal antibody directed to CD31. Angiogenesis is quantified by microvascular density counts within 20 high powered (200×) microscopic fields.

Hemoglobin content can be quantified as described previously (Schnaper, et al., J. Cell Physiol. 1993, 256: 235-246; Montesano, et al., J. Cell Biol. 1983, 97: 1648-1652; Stefansson, et al., J. Biol. Chem. 2000, 276: 8135-8141; Gigli, et al., J. Immunol. 1986, 100: 1154-1164). The Matrigel implants are snap frozen on dry ice and lyophilized overnight. The dried implants are resuspended in 0.4 ml of 1.0% saponin (Calbiochem) for one hour, and disrupted by vigorous pipetting. The preparations are centrifuged at 14,000 g for 15 minutes to remove any particulates. The concentration of hemoglobin in the supernatant is then determined directly by measuring the absorbency at 405 nm and compared to a standard concentration of purified hemoglobin. This method of quantification has been used successfully and has been shown to correlate with angiogenesis (Schnaper, et al., J. Cell Physiol. 1993, 256: 235-246; Montesano, et al., J. Cell Biol. 1983, 97: 1648-1652; Stefansson, et al., J. Biol. Chem. 2000, 276: 8135-8141; Gigli, et al., J. Immunol. 1986, 100: 1154-1164).

XIII. Methods of Assaying Cell Adhesion

Cell adhesion can be measured by methods known to those of skill in the art. Assays have been described previously, e.g. by Brooks, et al., J. Clin. Invest 1997, 99:1390-1398. The Examples below describes such an in vitro cell adhesion assay, in which cells are allowed to adhere to substrate (i.e., denatured collagen type-IV) on coated wells. Non-attached cells are removed by washing, and non-specific binding sites are blocked by incubation with BSA. The attached cells are stained with crystal violet, and cell adhesion is quantified by measuring the optical density of eluted crystal violet from attached cells at a wavelength of 600 nm.

XIV. Methods of Assaying Cell Migration

Assays for cell migration have been described in the literature, e.g., by Brooks, et al., J. Clin. Invest 1997, 99:1390-1398 and methods for measuring cell migration are known to those of skill in the art. In one method for measuring cell migration described herein in the Examples, membranes from transwell migration chambers are coated with substrate (here, thermally denatured collagen), the transwells washed, and non-specific binding sites blocked with BSA. Tumor cells from sub-confluent cultures are harvested, washed, and resuspended in migration buffer in the presence or absence of assay antibodies. After the tumor cells are allowed to migrate to the underside of the coated transwell membranes, the cells remaining on the top-side of the membrane are removed and cells that migrate to the under-side are stained with crystal violet. Cell migration is then quantified by direct cell counts per microscopic field.

XV. Methods of Assaying Tumor Growth

Tumor growth can be assayed by methods known to those of skill in the art, e.g., as described in (Xu, et al., J. Cell Biol 2001, 154:1069-1079). An assay for chick embryo tumor growth can be performed as follows: single cell suspensions of CS1 melanoma (5×10⁶ per embryo) or HT1080 fibrosarcoma (4×10⁵ per embryo) are applied in a total volume of 40 μl of RPMI to the CAMs of 10-day-old embryos (Brooks et al., 1998). Twenty four hours later, the embryos receive a single intravenous injection of purified Mab HUIV26 or control Mab (100 μg per embryo). Tumors are grown for 7 days, then resected and wet weights are determined. Experiments can be performed with five to ten embryos per condition.

Another method for assaying tumor growth makes use of the SCID mouse, as follows:

Subconfluent human M21 melanoma cells are harvested, washed, and resuspended in sterile PBS (20×10⁶ per ml). SCID mice are injected subcutaneously with 100 μl of M21 human melanoma cell (2×10⁶) suspension. Three days after tumor cell injection, mice are either untreated or treated intraperitoneally (100 μg/mouse) with either Mab HUIV26 or an isotype-matched control antibody. The mice are treated daily for 24 days. Tumor size is measured with calipers and the volume estimated using the formula V×L2×W/2, where V is equal to the volume, L is equal to the length, and W is equal to the width.

XVI. Methods for Administering Gene Product or Protein to a Patient

The dosage ranges for the administration of the product of a gene that is modulated by the specific binding of an antagonist to a cryptic ECM component epitope, or fragment thereof, depend upon the form of the gene product, and its potency, and are amounts large enough to produce the desired effect wherein angiogenesis, tumor metastasis, tumor growth, cell adhesion or cell migration are inhibited wherein the effect is favorable for treatment of an angiogenesis-dependent condition. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of the protein or polypeptide, e.g., a portion of the gene product having angiogenesis-, tumor metastasis-, tumor growth-, cell adhesion- or cell migration-inhibiting properties, sufficient to produce a measurable inhibition of angiogenesis, tumor metastasis, tumor growth, cell adhesion or cell migration in the tissue being treated or have an effect on an angiogenesis-dependent condition. Inhibition of these symptoms can be measured according to methods described herein, or by other methods known to one skilled in the art. Methods for assessing the effect on an angiogenesis-dependent condition will depend on the condition being treated, and for the particular condition, such methods will be known to those of skill in the art.

It is to be appreciated that the potency, and therefore an expression of a “therapeutically effective” amount can vary. However, as shown by the present assay methods, one skilled in the art can readily assess the potency of a gene product of this invention. Potency can be measured by a variety of means, including, but not limited to: the measurement of inhibition of angiogenesis in the CAM assay, in the in vivo rabbit eye assay, or in the in vivo chimeric mouse:human assay; the inhibition of tumor metastasis in the chick embryo model or in the murine model; the inhibition of cell adhesion in a cell adhesion assay; the inhibition of cell migration in a cell migration assay; or the inhibition of tumor growth in the chick embryo assay or the SCID mouse assay, all as described herein and in the literature and known to those of skill in the art, and the like assays.

A “therapeutically effective” amount of IGFBP-4 can be determined by prevention or amelioration of adverse conditions or symptoms of diseases, injuries or disorders being treated. For all the indications of use of IGFBP-4, the appropriate dosage will of course vary depending upon, for example, the tumor type and stage and severity of the disease disorder to be treated and the mode of administration. For example, tumor inhibition as a single agent may be achieved at a daily dosages from about to 0.1 mg/kg to 40 mg/kg body weight, preferably from about 0.2 mg/kg to about 20 mg/kg body weight of a binding protein of the invention. In larger mammals, for example, humans, as indicated daily dosage is from about 0.25 to about 5 mg/kg/day or about 70 mg per day for an average adult at a dose of 1 mg/kg/day conveniently administered parenterally, for example once a day. Dosage ranges for IGFBP-3 are described in U.S. Publication No. 20040127411, incorporated herein by reference.

The proteins or polypeptides of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, proteins or polypeptides of the invention can be administered intravenously, intraperitoneally, intramuscularly, subcutaneously, intracavity, transdermally, and can be delivered by peristaltic means.

Therapeutic compositions are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The present invention contemplates therapeutic compositions useful for practicing the therapeutic methods described herein. Therapeutic compositions of the present invention contain a physiologically tolerable carrier together with the protein or polypeptide as described herein, dissolved or dispersed therein as an active ingredient. In a preferred embodiment, the therapeutic protein or polypeptide composition is not immunogenic when administered to a mammal or human patient for therapeutic purposes.

As used herein, the terms “pharmaceutically acceptable,” “physiologically tolerable,” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, etc. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Examples of such additional liquid phases are glycerin, vegetable oils such as cottonseed oil, and water-oil emulsions.

In further embodiments, the invention enables any of the foregoing methods to be carried out in combination with other therapies such as, for example, treatment with another compound, e.g., an inhibitor of angiogenesis and tumor development processes (e.g., a monoclonal antibody that binds to the cryptic collagen epitope, HUIV26), chemotherapy or radiation therapy, or treatment with cytotoxic agents. Chemotherapeutic agents useful in the methods of the present invention include, e.g., taxanes (i.e., Taxol, Docetaxel, Paclitaxel), dacarbazine (DTIC), Adriamycin, Bleomycin, Gemcitabine, Cyclophosphamide, Oxaliplatin, Camptothecan, Ironotecan, Fludarabine, Cisplatin and Carboplatin.

An angiogenesis inhibitor may be administered to a patient in need of such treatment before, during, or after chemotherapy. It is also preferred to administer an angiogenesis inhibitor to a patient as a prophylaxis against metastases after surgery on the patient for the removal of solid tumors.

XVII. Methods of Detection

Modulation of the expression of IGFBP-4, TSP-1, Id-1, or p21^{CIP1} can be indicative of the effectiveness of the inhibition of angiogenesis, metastasis, and associated processes resulting from administration of an antagonist that specifically binds to a cryptic epitope of an ECM component.

In the detection methods of the present invention, levels of nucleic acids or proteins can be measured to confirm modulation of the expression levels of IGFBP-4, TSP-1, Id-1, and p21^{CIP1}. Nucleic acid and protein levels can be determined using techniques known to those of skill in the art and described in the literature. For example, nucleic acids can be studied using Real Time quantitative RT-PCR. Primer sequences useful for detecting IGFBP-4, TSP-1, Id-1, and p21^{CIP1}, are given below in the Examples, and additional primer sequences for these genes can be determined by sequence analysis methods and using software as known and commonly used by those of skill in the art.

In addition to PCR techniques, other methods for detecting and quantifying nucleic acids are well known to those skilled in the art and have been described in the literature, including hybridization methods, e.g., Southern blotting, Northern blotting, etc. Amplification techniques, including PCR, can be used prior to analysis using any of these methods.

As described herein, Enzyme Linked immunosorbent Assay (ELISA) and Western Blot analysis, as well as radioimmunoassay immunoprecipitation, can be used for measuring levels of TSP-1, IGFBP-4, Id-1, and P21^{CIP} proteins in the detection methods of the invention.

XVIII. Cell Lines

The methods of the present invention can be practiced using a number of cell lines which are obtained and maintained according to methods known to those of skill in the art. For example, murine B16F10 melanoma cell line was obtained from ATCC (Rockville, Md.). Tumor cells were maintained in Dulbecco's Modified Eagles Medium (DMEM) (Gibco Grand Island N.Y.) supplemented with 10% Fetal Bovine Serum (FBS) (Hyclone, Logan Utah), 1.0% Sodium Pyruvate, Glutamate and Pen-Strep (Gibco, Grand Island N.Y.). Cells were maintained as sub-confluent cultures before use and harvested with trypsin-EDTA (Gibco, Grand Island N.Y.

Cell lines described herein have been previously described, as follows: ECV and ECVL in Brooks, et al., Cell 1998, 92:391-400; M21 and M21L in Montgomery, et al., Proc. Natl. Acad. Sci. USA 1994, 91:8856-8860, and; CS1 and b3CS1 in Brooks, et al., Cell 1996, 85:683-693.

The patents and publications cited herein reflect the level of skill in this field and are hereby incorporated by reference in their entirety to the same extent as if each was specifically and individually indicated to be incorporated by reference.

### EXAMPLES

The present invention is further illustrated by the following examples, which should not be construed as limiting in any way.

### Example I

### Mab HUIV26 Inhibits Tumor Cell Interactions with Denatured Type-IV Collagen

In vitro cell adhesion assays indicated that malignant tumor cells utilize the HUIV26 cryptic epitope in attaching to denatured collagen type-IV.

Highly metastatic B16F10 melanoma cells were allowed to adhere to denatured collagen type-IV coated wells in the presence or absence of Mab HUIV26 or an isotype-matched control antibody. Cell adhesion assays were carried out as described previously with some modifications (Brooks, et al., J. Clin. Invest 1997, 99:1390-1398). Forty-eight-well non-tissue culture plates were coated with thermally denatured collagen (10.0 μg/ml) for 12 hours at 4° C. The plates were next washed with PBS and non-specific binding sites were blocked by incubation with 1.0% BSA in PBS for 1 hour at 37° C. Tumor cells (B16F10) from sub-confluent cultures were harvested, washed and resuspended in adhesion buffer containing RPMI 1640, 1 mM MgCl₂, 0.2 mM MnCl₂ and 0.5% BSA in the presence or absence of function blocking antibodies (100 μg/ml) or isotype-matched control antibody. Tumor cells were added to the coated plates in a total volume of 200 μl and allowed to attach for 15 to 30 minutes. Non-attached cells were removed by washing, and attached cells were stained with crystal violet as described previously (Brooks, et al., J. Clin. Invest 1997, 99:1390-1398). Cell adhesion was quantified by measuring the optical density of eluted crystal violet from attached cells at a wavelength of 600 nm (Brooks, et al., J. Clin. Invest 1997, 99:1390-1398).

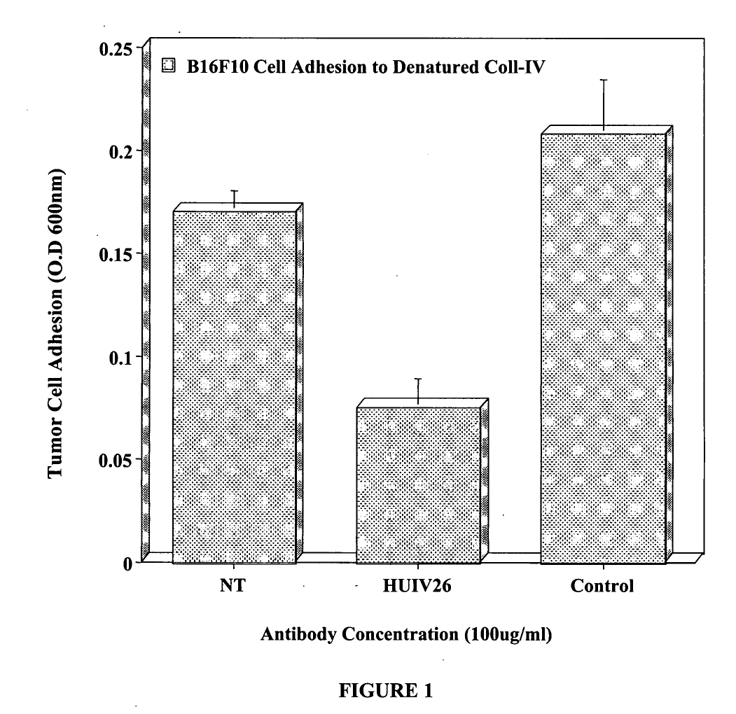

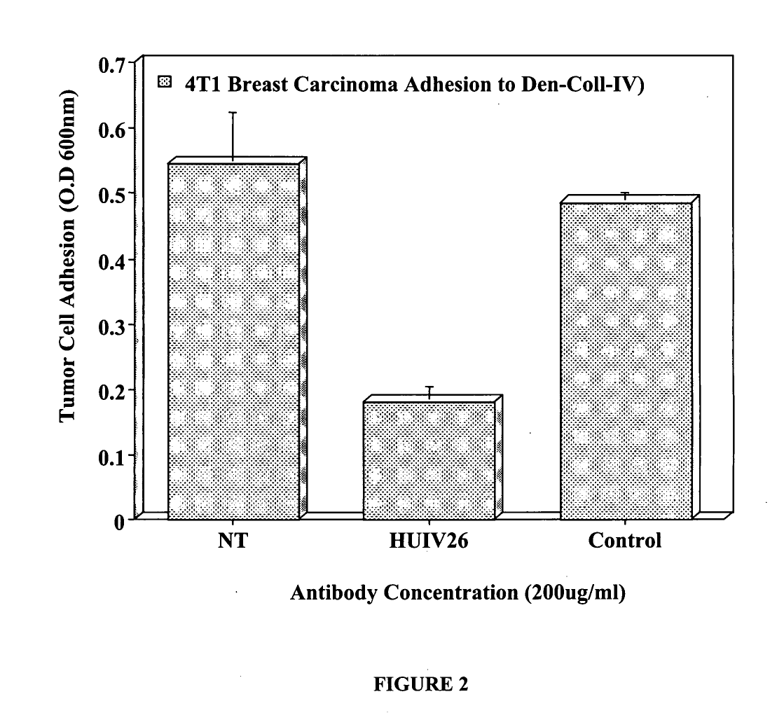

As shown in FIG. 1, B16F10 melanoma cells readily attached to denatured collagen type-IV. In contrast, Mab HUIV26 specifically inhibited adhesion of B16F10 cells to denatured collagen type-IV by approximately 50% as compared to either no treatment (NT) or treatment with an isotype matched control antibody (Control). In addition, Mab HUIV26 had no effect on tumor cell adhesion to other ECM proteins in either its intact or denatured forms (data not shown) (Xu, et al., J. Cell Biol. 2001, 154:1069-1079). Similar results were obtained with the metastatic breast carcinoma cell line 4T1, suggesting that the effects of Mab HUIV26 was not restricted to a single cell type (FIG. 2).

### Example II

### Mab HUIV26 Inhibits Tumor Cell Migration on Denatured Type-IV Collagen

Examination of the effects of Mab HUIV26 on malignant tumor cell migration in vitro were examined using cell migration assays showed that melanoma cells readily migrate on denatured collagen type-IV.

Protocols for cell migration assays have been described previously (Brooks, et al., J. Clin. Invest 1997, 99:1390-1398). Similar methods were used here with some modifications. Membranes (8.0 μm pore size) from transwell migration chambers were coated with thermally denatured collagen (10.0 μg/ml) for 12 hours at 4° C. The transwells were next washed with PBS and non-specific binding sites were blocked by incubation with 1.0% BSA in PBS for 1 hour at 37° C. B16F10 tumor cells from sub-confluent cultures were harvested, washed and resuspended in migration buffer containing RPMI 1640, 1 mM MgCl₂, 0.2 mM MnCl₂ and 0.5% BSA in the presence or absence of HUIV26 function-blocking antibodies (100 μg/ml) or isotype-matched control antibodies. Tumor cells were allowed to migrate to the underside of the coated transwell membranes for 2 to 4 hours. Tumor cells remaining on the top-side of the membrane were removed and cells that had migrated to the under side were staining with crystal violet as described previously (Brooks, et al., J. Clin. Invest 1997, 99:1390-1398). Cell migration was quantified by direct cell counts per microscopic field.

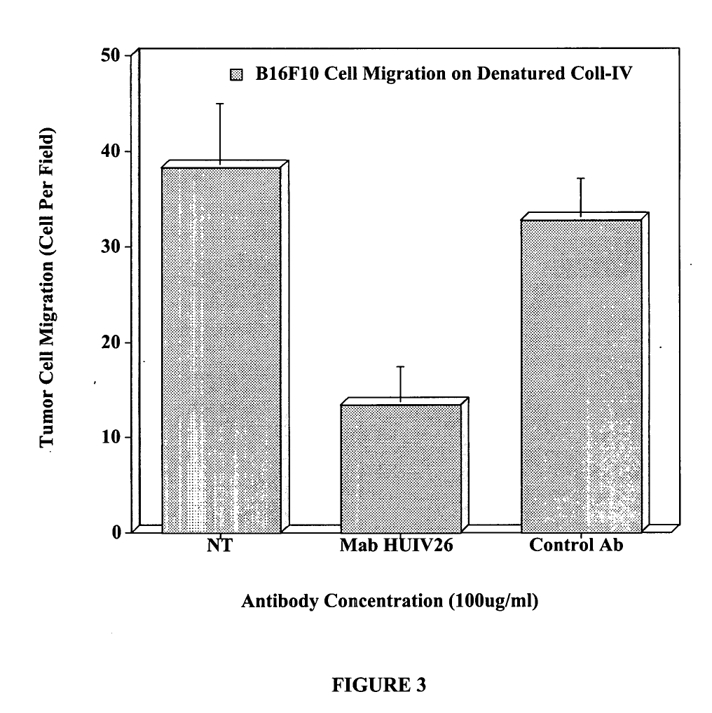

As shown in FIG. 3, B16F10 melanoma cells readily migrate on denatured collagen type-IV. In contrast, migration of B16F10 tumor cells were inhibited by approximately 50% as compared to controls.

### Example III

### Mab HUIV26 Dose Dependently Inhibits B16F10 Experimental Metastasis in the Chick Embryo Model

A rapid experimental metastasis assay to study the potential role of the HUIV26 epitope in metastasis was established. This assay was used to demonstrate that Mab HUIV26 inhibits experimental metastasis of tumor cells to the lungs of chick embryos.

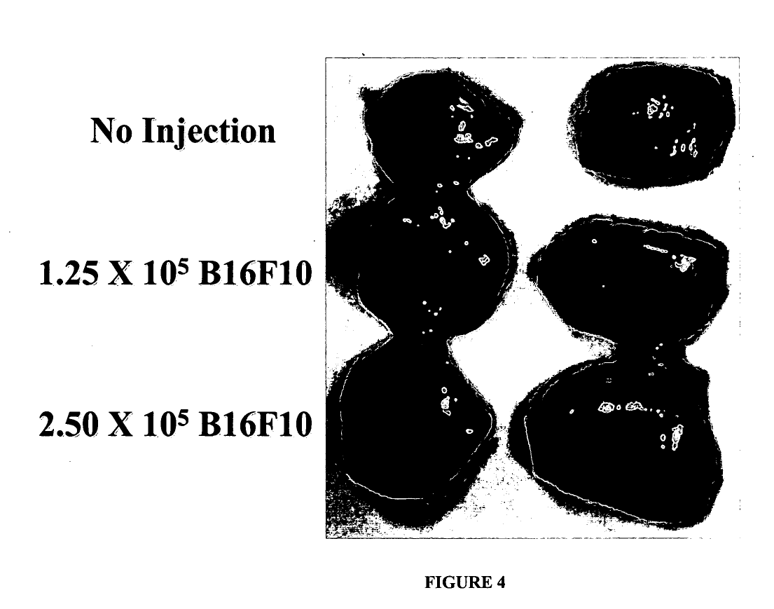

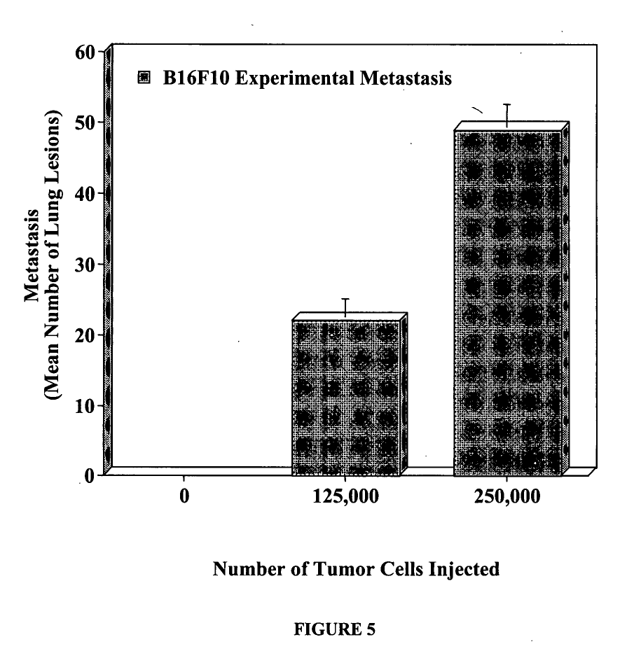

The chick embryo model was used in conjunction with metastatic B16F10 melanoma cells. Sub-confluent B16F10 melanoma cells were resuspended at a final concentration of 2.5×10⁶ cells per ml in sterile PBS. Twelve-day-old chick embryos were injected intravenously with 100 μl of B16F10 cell suspension and the embryos were allowed to incubate for a total of 7 days. At the end of the 7-day incubation period, the embryos were sacrificed and the lungs were resected and analyzed. As shown in FIG. 4, intravenous injections of increasing concentrations of B16F10 melanoma cells resulted in the dose dependent formation of numerous discrete melanotic lesions, which could be readily seen on the surface of the chick lungs. To quantify the experimental metastasis, the chick lungs were removed and the total number of discrete independent foci was counted on both lobes for each lung and metastasis was expressed as the mean number of discrete B16F10 foci per lung per group. As shown in FIG. 5, a concentration-dependent increase in the mean number of lung lesions was observed following injections of increasing number of B16F10 cells.

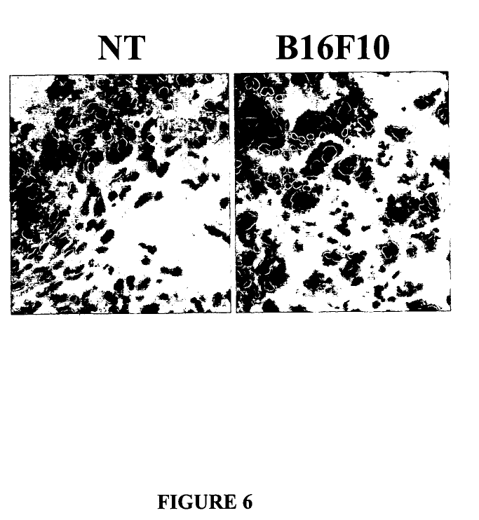

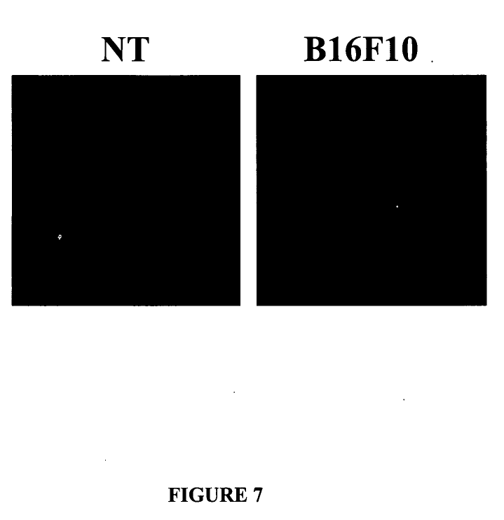

To confirm the presence of the tumor cells within the chick lungs, histological analysis was performed. Frozen sections from either normal lungs or lungs from embryos injected with B16F10 cells were stained with Hematoxylin and Eosin. As shown in FIG. 6, top panel, lungs from untreated embryos exhibited normal tissue architecture and stromal organization. In contrast, numerous large tumor cells with irregular nuclei were easily seen scattered throughout the lung tissue from embryos injected with B16F10 cells. These B16F10 cells were detected as scattered individual cells and groups of clustered tumor cells organized into discrete tumor foci. To confirm the presence of the melanoma cells immunologically, lung sections were analyzed for the expression of the melanoma-associated antigen MART-1 (Berset et al., Int. J. Cancer 2001, 95: 73-77). As shown in FIG. 7, no specific expression of the MART-1 antigen was detected in the lungs from untreated control embryos (left panel). In contrast, tumor cells within the lungs derived from embryos injected with the B16F10 cells stained positive for the MART-1 antigen (right panel). Taken together, these findings confirm the suitability of this model to assess the potential anti-metastatic effects of Mab HUIV26.

The effects of Mab HUIV26 on B16F10 experimental metastasis were tested in this model.

Twelve-day-old fertilized chick eggs were obtained from SPAFAS (North Franklin, Conn.) and maintained in a 48-place tabletop egg incubator (Lyon Electric, Chula Vista Calif.) as described previously (Brooks et al., Meth. Mol. Biol. 1999, 129:257-269; Testa et al., Cancer Res 1999, 59: 3812-3820). Prominent blood vessels were visualized through the eggshell of the 12-day-old chick embryos with the aid of an egg candle (Brooks et al., Meth. Mol. Biol 1999, 129:257-269; Testa et al., Cancer Res. 1999, 59: 3812-3820). The area of the outer egg shell where prominent blood vessels are located close to inner shell surface was swabbed with 70% ethanol and a small window was cut through the egg shell with a hobby grinding wheel (Dremel Emerson Electric Co., Racine, Wis.). The embryos were returned to the incubator until tumor cells were prepared for injection.

Subconfluent B16F10 cells were harvested, washed and resuspended in sterile PBS in the presence of absence of Mab HUIV26 or an isotype-matched control antibody, and harvested with trypsin EDTA. Tumor cells were washed with serum containing DMEM and resuspended in sterile PBS at concentrations ranging from 0.5 to 5.0×10⁶ per ml. Next, the small windows cut through the egg shell were carefully removed and a drop of mineral oil was added to the shell membrane to enhance visualization of the underlying blood vessel (Brooks et al., Meth. Mol. Biol 1999, 129:257-269; Testa et al., Cancer Res 1999, 59: 3812-3820). Tumor cell suspensions were injected intravenously in a total volume of 100 μl per embryo. The embryos were allowed to incubate undisturbed for a total of 7 days. To quantify experimental B16F10 lung metastasis, embryos were sacrificed at day 19 and both lobes of the chick lungs were dissected. The lungs were analyzed with the aid of a stereo microscope set at a defined magnification. The total number of isolated and discrete pigmented lung surface lesions was carefully counted on each side of each lobe for each embryo. A typical experiment would include at least 8-10 embryos per condition. Experimental metastasis was described as the mean number of surface B16 melanoma lesions per lung per experimental condition.

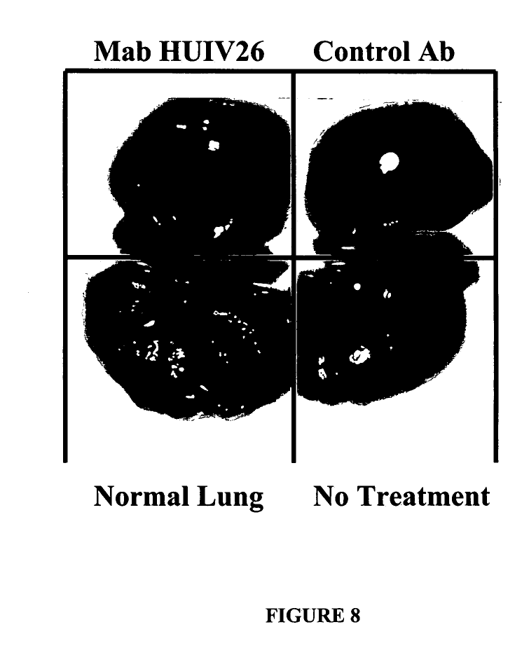

As shown in FIG. 8, injection of untreated B16F10 melanoma cells resulted in the formation of extensive lung foci. In contrast, lungs from chick embryos treated with Mab HUIV26 exhibited a dramatic reduction in B16F10 lung surface lesions.

Histological examination of the lungs confirmed a reduction in infiltration of B16F10 melanoma cells into the lung tissue. Lungs from chick embryos or mice were dissected, and embedded in OTC, snap frozen and 4.0 μm sections were cut with a cryostat as described previously (Brooks, et al., Cell 1996, 85:683-693; Brooks, et al., Science 1994, 264:569-571). For histological analyses, frozen sections of lung tissue were fixed in 10% formalin and stained with hematoxylin and eosin. Immunofluoresence analysis was performed as previously described with some modifications (Brooks, et al., Cell 1996, 85:683-693; Brooks, et al., Science 1994, 264:569-571). Lung sections (4.0 μm) were fixed for 30 seconds in 50% methanol and 50% acetone. Next, tissue sections were incubated for 2.0 minutes in 0.2% Triton X-100 in PBS. The tissue sections were washed 3 times and incubated with 2.5% BSA in PBS to block non-specific binding sites. Monoclonal antibody A103 (Anti-MART-1 antigen) was diluted in 2.5% BSA in PBS to a final concentration of 10 μg/ml and 100 μl was added to the tissue sections. Tissues were incubated for a total of 2 hours at 37° C. The tissues were next washed 5 times with PBS for 5 minutes each, followed by incubation with 1:300 dilution of rhodamine conjugated goat anti-mouse secondary for 1 hour. Finally, the tissues were washed as before, mounted in anti-fade medium and sealed with clear nail polish.

To quantify the effects of Mab HUIV26 on B16F10 experimental metastasis, the number of B16F10 surface lesions was counted for each lung. Statistical analysis of experimental data was analyzed using unpaired student T-test. P values of less than 0.05 were considered significant.

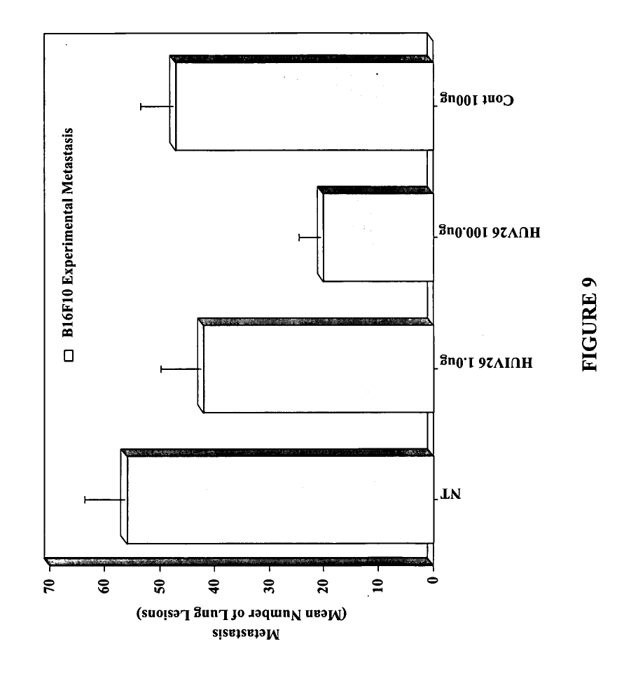

As shown in FIG. 9, in the presence of Mab HUIV26 (100 μg), the mean number of B16F10 lung foci was significantly (P<0.001) reduced by approximately 65% as compared to no treatment or to treatment with isotype-matched control antibody, while 1.0 μg of Mab HUIV26 per embryo exhibited little effect. Taken together, these findings suggest that Mab HUIV26 potently inhibits experimental metastasis of B16F10 to the lungs of chick embryos.

### Example IV

### Mab HUIV26 Inhibits B16F10 Experimental Metastasis in Mice

To assay the anti-metastatic activity of Mab HUIV26 and to examine its effects in a second experimental system, a murine model was used. The assay was carried out essentially as described by Vantyghem, et al., Cancer Res 2003, 63:4763-4765 with some modifications. Female Balb/c mice were injected intravenously (100 μl) with B16F10 melanoma cells (2×10⁵) in the presence or absence of Mab HUIV26 or an isotype matched control. Following injection of the tumor cells, the mice were treated daily by intraperitoneal injection with either Mab HUIV26 or control antibody (100 μg) in a total volume of 100 μl of sterile PBS for 7 days. At the end of the 7-day treatment period the mice were sacrificed and the lungs were removed for analysis. To quantify experimental B16F10 lung metastasis, lungs were dissected and place in 35 mm culture plates. The lungs were analyzed with the aid of a stereomicroscope set at a defined magnification (30×). The total number of isolated and discrete pigmented lung surface lesions was carefully counted on each lobe for each specimen. Experimental metastasis is described as the mean number of surface tumor lesions per lung per experimental condition. Presence of tumor lesions within the lungs was confirmed by histological analysis, as described above.

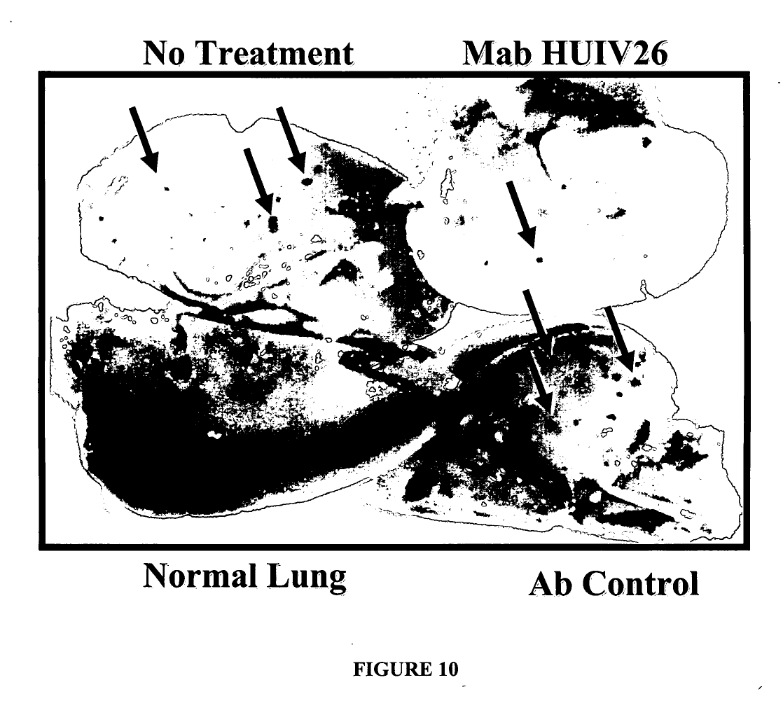

Extensive B16F10 melanoma lesions could be detected on the surface of the murine lungs while a significant reduction in tumor lung lesions were observed on lungs from mice treated with Mab HUIV26 (FIG. 10). To quantify the anti-metastatic effects of Mab HUIV26, the number of lung surface lesions was counted. Statistical analysis of experimental data was analyzed using unpaired student T-test. P values of less than 0.05 were considered significant.

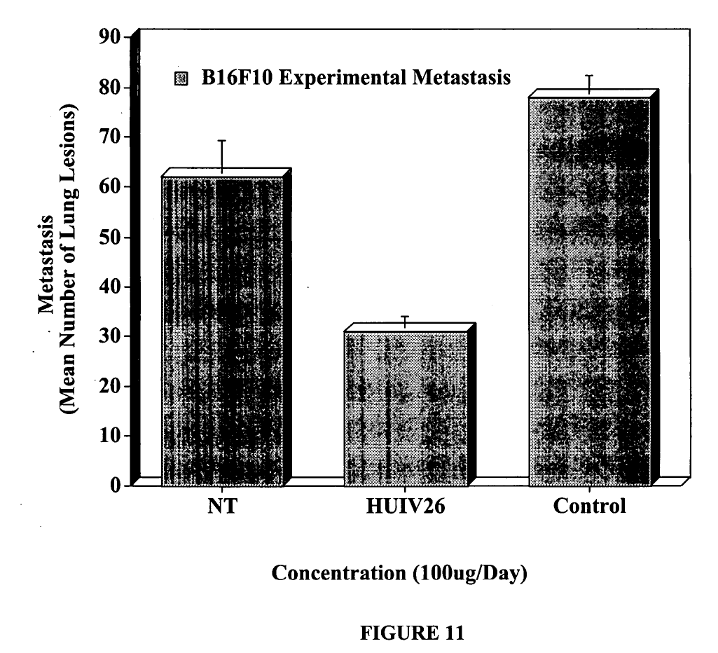

As shown in FIG. 11, Mab HUIV26 significantly (P<0.05) inhibited B16F10 experimental metastasis by approximately 50% as compared to either no treatment or treatment with an isotype matched control antibody. Collectively, these data suggest that tumor cell interactions with the HUIV26 epitope may contribute to the regulation of metastasis and that blocking cellular interactions with the HUIV26 epitope may represent a novel approach to control the spread of malignant tumor cells to distant sites.

### Example V

### Inhibition of Tumor Cell Interactions with the HUIV26 Cryptic Site Enhances Expression of P21^{CIP1} RNA

Expression levels of differential cDNA array analysis of B16F10 tumor cells treated with Mab HUIV26 suggested a significant increase in the expression of several genes including the cyclin dependent kinase inhibitor P21^{CIP1}.

An Affymetrix™-based differential cDNA array analysis was performed using B16F1 tumor cells treated or not treated with Mab HUIV26. Non-tissue culture treated dishes were coated overnight with 100 μg/ml of denatured collagen IV in PBS. The next morning the plates were washed and incubated in blocking solution (1% BSA in PBS) for approximately 30 minutes.

Tumor cells (7×10⁶) were resuspended in serum-free media and added to each plate in the presence or absence of Mab HUIV26 or a control isotype-matched IgM antibody (100 μg/ml). The cells were allowed to incubate for a total of 12 hours. Following the 12-hour incubation period, the cells were harvested and the RNA was isolated using both a TRIzol reagent and the Qiagen Rneasy Mini Protocol for RNA Cleanup. After RNA extraction, the amount and quality of RNA was quantified utilizing a spectrophotomer, and 5-8 μg of total RNA was utilized to synthesize double-stranded cDNA.

The first cDNA strand was obtained using a reaction mixture containing a T7-(dT)24 Primer, 1× First Strand Buffer, 0.1M DTT and 10 mM dNTP mix in addition to the extracted RNA. The tubes were incubated at 42° C. for approximately 1.5 hours.

For the second strand cDNA synthesis, a 1× Second Strand Buffer, 10 mM dNTP mix, 10 U/ml of E. coli DNA Ligase, 10 U/ml of DNA Polymerase I and RNaseH were added and allowed to incubate at 16° C. for 2.5 hours. Following the incubation period, T4 DNA Polymerase was added and the tubes were incubated for 5 minutes and stored at −80° C. The final double-stranded cDNA product was cleaned utilizing phenol extraction and ethanol precipitation. Next, the synthesized cDNA was converted to cRNA and labeled with biotin labeled ribonucleotides in a reaction mixture that also included HY Reaction Buffer, 10×DTT, Rnase Inhibitor Mix and 20× RNA Polymerase. The final cRNA product was cleaned utilizing the Qiagen Rneasy Mini Protocol for RNA Cleanup and 15 μg of cRNA was fragmented and hybridized to a U95Av2 chip.

Relative expression levels of P21^{CIP1} were then assessed by both real time RT-PCR and Western Blot analysis. Tumor cells (B16F10) were allowed to interact with denatured collagen type-IV in the presence or absence of Mab HUIV26 or an isotype-matched control antibody, and mRNA and whole cell lysates were prepared.

Real Time quantitative RT-PCR was carried out essentially as previously described with some modifications (Livak et al., Method 2001, 25:402-408). Total RNA was isolated using RNeasy miniprep columns (Qiagen, Valencia Calif.) according to the manufacturer's instructions. Total RNA (1 μg) was reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection was carried out using an ABI Prism 7900 Sequence Detection System. Reactions were carried out in microAmp 96 well reaction plates. Primers and probes were designed using Primer 3 version 2 and ENSEMBL software (Promega).

The primer sets used to detect P21^{CIP1} were:

SEQ ID NO: 1)

5′-CTTGTCGCTGTCTTGCACTC-3′ (forward;
and

SEQ ID NO: 2)

5′-AATCTGTCAGGCTGGTCTGC-3′ (reverse;.

The primers used to detect control gene β2-macroglobulin were:

SEQ ID NO: 3)

5′-AAAGATGAGTATGCCTGCCG-3′ (forward;
and

SEQ ID NO: 4)

5′-CCTCCATGATGATGCTGCTTACA-3′ (reverse;.

cDNA from samples are labeled with SYBR Green (Roche) and real time PCR was run using a Light Cycler (NYU Genomic Core Services). Quantification of data was performed using Light Cycler real time PCR analysis software package 3.5 (Roche).

Fold induction was calculated using methods described by Livak et. al, 2001. Amplification products utilized through Sybergreen detection were initially checked by electrophoresis on ethidium bromide stained agarose gels. The estimated size of the amplified products matched the calculated size for transcript by visual inspection.

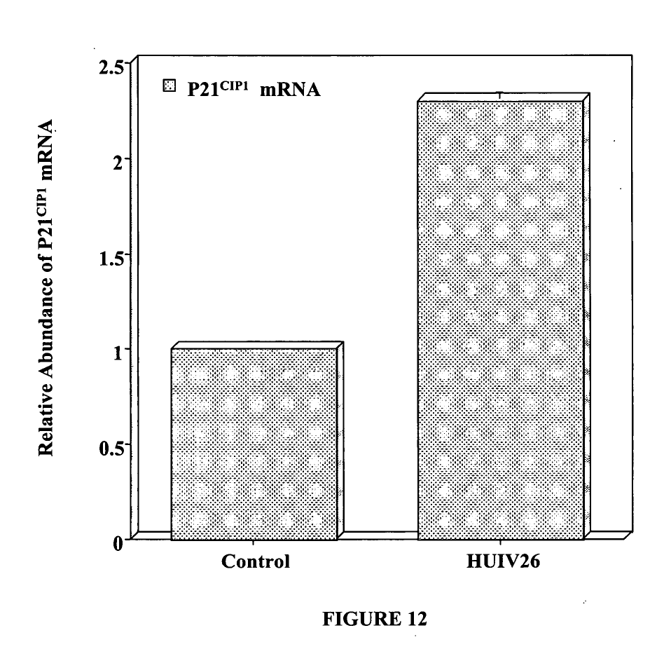

As shown in FIG. 12, the relative level of P21^{CIP1} mRNA was increased by approximately 2.3 fold as compared to an isotype-matched non-specific control antibody. Moreover, no changes in the relative levels of control genes β-actin or β2-macroglobulin were observed following treatment of B16F10 cells with Mab HUIV26 (data not shown).

### Example VI

### Inhibition of Tumor Cell Interactions with the HUIV26 Cryptic Site Enhances Expression of P21^{CIP1} Protein

Western Blotting experiments indicated an approximately 2-fold increase in the expression of P21^{CIP1} mRNA in tumor cells incubated with Mab HUIV26.

Western blot analysis was performed by coating non-tissue culture treated plates with denatured collagen type-IV (10.0 μg/ml). Equal numbers of tumor cells (B16F10) from sub-confluent cultures were harvested, washed and added to the coated plates in the presence or absence of Mab HUIV26 or an isotype-matched control antibody and allowed to incubate in 1.0% serum-containing medium. Cells were harvested, washed and lysed in 1.0% Triton X-100 buffer containing 300 mM NaCl, 50 mM Tris (pH 7.0) and 1× protease inhibitor cocktail. Equal amounts (25 μg/lane) of tumor cell lysates were separated by SDS Page and transferred to nitrocellulose membranes. Membranes were probed by incubation with either polyclonal antibodies directed to P21^{CIP1} or actin (Santa Cruz) as described previously (Brooks, et al., Cell 1998, 92: 391-400; Petitclerc, et al., Cancer Res. 1999, 59: 2724-2730. Western blots were visualized by a chemiluminescence detection system (Amersham Life Sciences).

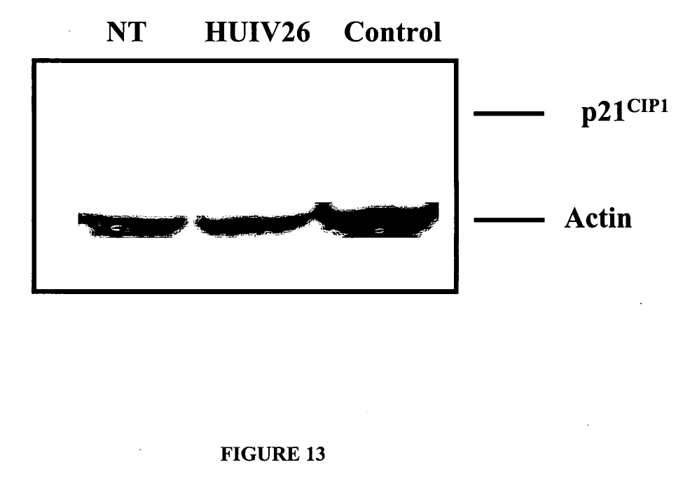

As shown in FIG. 13, incubation of B16F10 tumor cells plated on denatured collagen type-IV with Mab HUIV26 resulted in an approximately 2-fold increase in expression of P21^{CIP1} as compared to either no treatment or treatment with an isotype-matched control non-specific antibody. Importantly, no change in the relative levels of the control protein actin was observed under the different experimental conditions. These novel findings suggest that tumor cell interactions with the HUIV26 cryptic epitope within collagen type-IV may play a unique role in regulating expression of specific CDK inhibitors. Interestingly, alterations in expression cell cycle control proteins as well as modulation of tumor cell adhesion and migration are thought to play important roles in tumor cell metastasis.

### Example VII

### Inhibition of Cellular Interactions with the HUIV26 Cryptic Collagen Epitope Enhances Expression of TSP-1 RNA

Differential cDNA array analysis showed increased expression of TSP-1 in tumor cells treated with Mab HUIV26 or Mab LM609, and in HUVECS treated with Mab HUIV26. RT-PCR experiments showed that inhibiting cellular interactions with the HUIV26 cryptic epitope increased expression of TSP-1 7-fold. M21 cells were allowed to interact with denatured collagen type-IV in the presence or absence of Mab HUIV26, Mab LM609, or an isotype matched control antibody for 12 hours. Following the incubation period, the cells were harvested and RNA isolated. The relative levels of TSP-1 RNA were examined by real-time PCR, as described above.

The following human-specific real time PCR primer pairs were used to detect TSP-1:

(SEQ ID NO: 5)

5′-TCCAAAGCGTCTTCACCAG-3′
and

(SEQ ID NO: 6)

5′-GAGACAGCCTTTGTTCCTGAG-3′.

The primers used to detect control gene β2-macroglobulin were:

SEQ ID NO: 3)

5′-AAAGATGAGTATGCCTGCCG-3′ (forward;
and

SEQ ID NO: 4)

5′-CCTCCATGATGATGCTGCTTACA-3′ (reverse;.

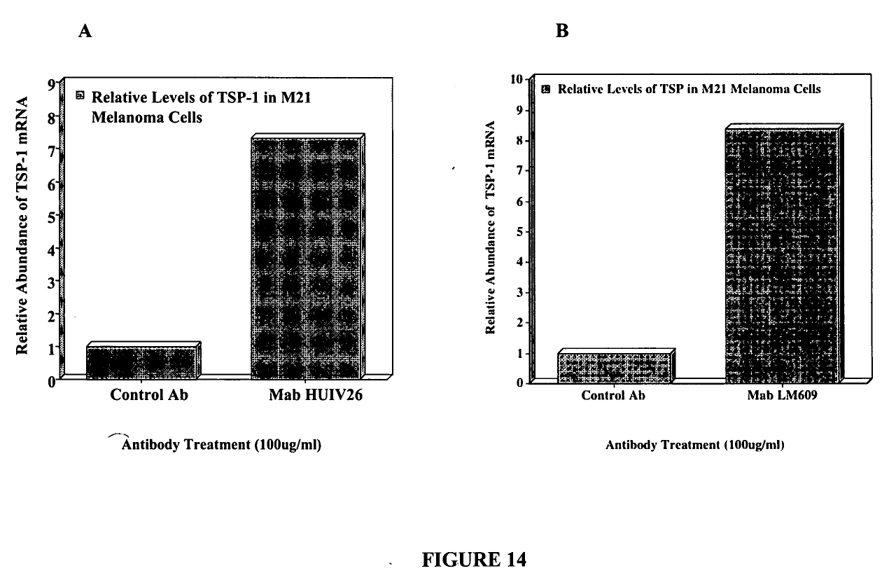

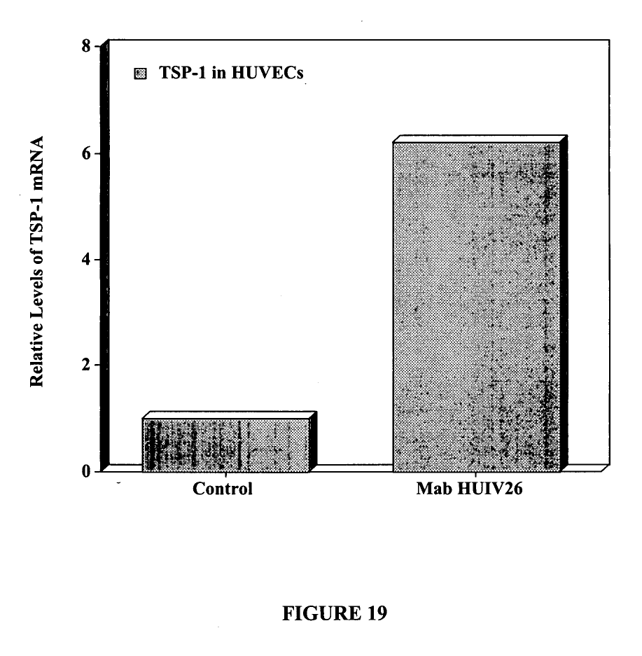

As shown in FIG. 14A, incubation of M21 cells with Mab HUIV26 resulted in an approximately 7-fold increase in the relative levels of TSP-1 RNA as compared to an isotype-matched control antibody. FIG. 19 shows that TSP-1 mRNA levels increased by about 6-fold when HUVECs were incubated in the presence of Mab HUIV26. Furthermore, FIG. 14B shows that the relative level of TSP-1 was elevated by approximately 8-fold in cells treated with the anti-αᵥβ₃-specific Mab LM609 as compared to an isotype-matched control antibody. These findings indicate that blocking cellular interactions with an αvβ3 ligand (HUIV26 cryptic epitope) enhances expression of endogenous angiogenesis inhibitors.

### Example VIII

### Inhibition of Cellular Interactions with the HUIV26 Cryptic Collagen Epitope Enhances Expression of IGFBP-4 RNA

Differential cDNA array analysis suggested increased expression of IGFBP-4 in tumor cells and HUVECs treated with Mab HUIV26. M21 cells were allowed to interact with denatured collagen type-IV in the presence or absence of Mab HUIV26 or an isotype matched control antibody for 12 hours. Following the incubation period, the cells were harvested and RNA isolated. The relative levels of IGFBP-4 RNA were examined by RT-PCR, as described above.

The following human-specific real time PCR primer pairs were used to detect IGFBP-4:

(SEQ ID NO: 7)

5′-CCTGCACACACTGATGCAC-3′
and

(SEQ ID NO: 8)

5′-GTCTCGAATTTTGGCGAAGT-3′.

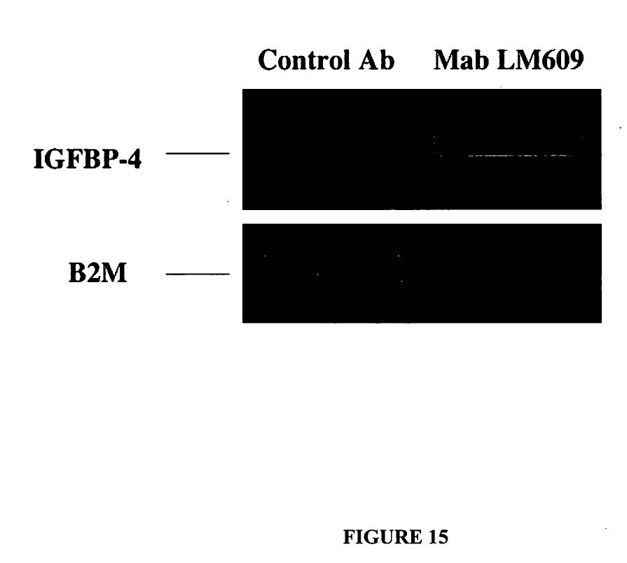

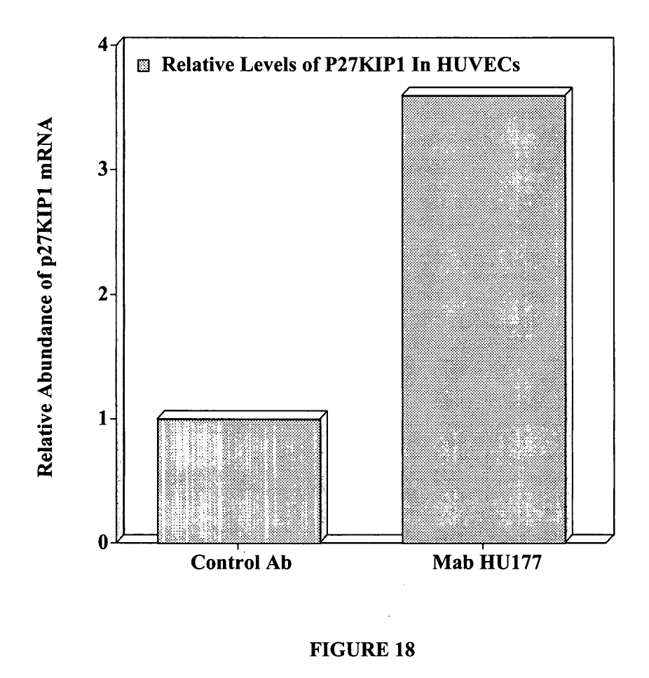

As shown in FIG. 15, incubation of M21 cells with Mab HUIV26 resulted in an approximately 115-fold increase in the relative levels of IGFBP-4 RNA as compared to isotype-matched controls. Furthermore, incubation of HUVECs with Mab HUIV26, as compared to isotype-matched controls, resulted in a greater than 10-fold increase in the relative levels of IGFBP-4 mRNA (FIG. 18). These findings provide further evidence that blocking cellular interactions with a αvβ3 ligand (HUIV26 cryptic epitope) enhances expression of endogenous angiogenesis inhibitors.

### Example IX

### Id-1 is Downregulated When Cellular Interactions with the HUIV26 Epitope are Inhibited

Differential cDNA array analysis showed a reduction of Id-1 expression in tumor cells lacking αvβ3. We examined the relative levels of Id-1 in M21 cells seeded on denatured collagen type-IV and incubated in the presence or absence of Mab HUIV26 or an isotype-matched control antibody for 12 hours. Following the incubation period cells were harvested and RNA isolated. The relative level of Id-1 was examined by real-time PCR, performed as described above.

The following human-specific real time PCR primer pairs were used to detect Id-1:

| | |
|---|---|
| 5′-ACGCCTCAAGGAGCTGGT-3′, | (SEQ ID NO: 9) |
| and | |
| 5′-CGCTTCAGCGACACAAGAT-3′. | (SEQ ID NO: 10) |

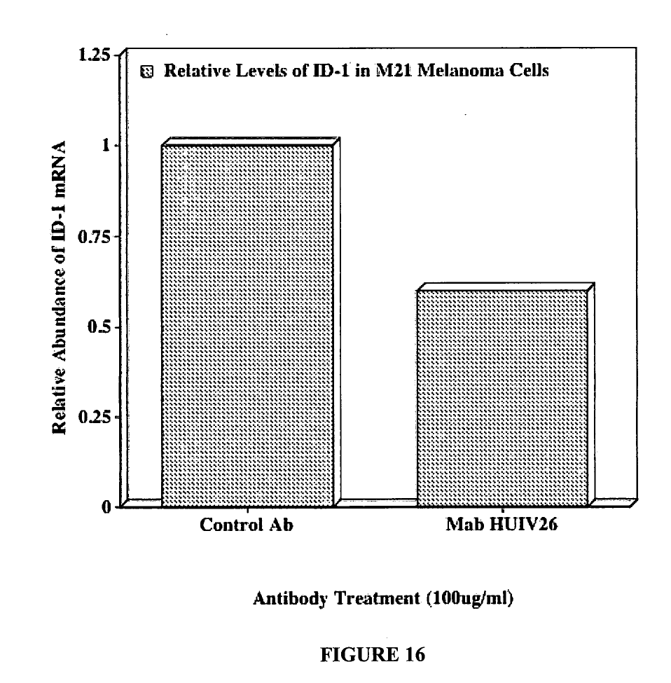

As shown in FIG. 16, incubating M21 cells in the presence of Mab HUIV26 resulted in a nearly 2-fold decrease in the relative levels of Id-1 as compared to isotype-matched control antibody treatment. These findings are consistent with cDNA array analysis and with the possibility that the αvβ3-dependent regulation of TSP-1 may involve altered expression of Id-1.

### Example X

### Peptide Inhibition of Tumor Cell Interactions with the HUIV26 Cryptic Site Enhances Expression of Certain Genes

To evaluate the effect of inhibiting the HUIV26 cryptic collagen epitope using the SLK- and CLK-Peptides, an Affymetrix™-based differential cDNA array analysis is performed using B16F10 tumor cells treated or not treated with SLK- or CLK-peptide. Non-tissue culture treated dishes are coated overnight with 100 μg/ml of denatured collagen IV in PBS. The next morning the plates are washed and incubated in blocking solution (1% BSA in PBS) for approximately 30 minutes. Tumor cells (7×10⁶) are resuspended in serum free media and added to each plate in the presence or absence of CLK-Peptide, SLK-Peptide or a control peptide. The cells are allowed to incubate for a total of 12 hours.

Following the 12-hour incubation period, the cells are harvested and the RNA is isolated using both a TRIzol reagent and the Qiagen Rneasy Mini Protocol for RNA Cleanup. After RNA extraction, the amount and quality of RNA is quantified utilizing a spectrophotomer. 5-8 μg of total RNA is utilized to synthesize double-stranded cDNA. The first cDNA strand is obtained using a reaction mixture containing a T7-(dT)₂₄ Primer, 1× First Strand Buffer, 0.1M DTT and 10 mM dNTP mix in addition to the extracted RNA. The tubes are incubated at 42° C. for approximately 1.5 hours. For the second strand cDNA synthesis, a 1× Second Strand Buffer, 10 mM dNTP mix, 10 U/ml of E. coli DNA Ligase, 10 U/ml of DNA Polymerase I and RNaseH are added and allowed to incubate at 16° C. for 2.5 hours.

Following the incubation period, T4 DNA Polymerase is added and the tubes are again incubated for 5 minutes and stored at −80° C. The final double-stranded cDNA product is cleaned utilizing phenol extraction and ethanol precipitation. Next, the synthesized cDNA is converted to cRNA and labeled with biotin labeled ribonucleotides in a reaction mixture that also includes HY Reaction Buffer, 10×DTT, Rnase Inhibitor Mix and 20×RNA Polymerase. The final cRNA product is cleaned utilizing the Qiagen Rneasy Mini Protocol for RNA Cleanup and 15 μg of cRNA is fragmented and hybridized to a U95Av2 chip.

Expression levels of differential cDNA array analysis of B16F10 tumor cells treated with CLK-Peptide or SLK-Peptide suggest a significant increase in the expression of certain genes.

Relative expression levels of genes are assessed by both real time quantitative RT-PCR and Western Blot analysis. Tumor cells (B16F10) are allowed to interact with denatured collagen type-IV in the presence or absence of the SLK-peptide, CLK-peptide, or control peptide. Whole cell lysates are prepared from the various cell samples.

Real Time quantitative RT-PCR is carried out essentially as described with some modifications (Livak et al., Method 2001, 25:402-408). Total RNA is isolated using RNeasy miniprep columns (Qiagen, Valencia Calif.) according to the manufacturer's instructions. Total RNA (1 μg) is reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection is carried out using an ABI Prism 7900 Sequence Detection System. Reactions are carried out in microAmp 96 well reaction plates. Primers and probes are designed using Primer 3 version 2 and ENSEMBL software (Promega).

Incubation of cells with SLK-peptide or CLK-peptide results in a significant increase in the relative levels of certain RNAs and significant decrease in other RNAs as compared to incubation with control peptide.

Western blot analyses indicate a corresponding increase in protein levels. Western blotting was performed as previously described, by coating non-tissue culture treated plates with denatured collagen type-IV (10.0 μg/ml). Equal numbers of tumor cells (B16F10) from sub-confluent cultures are harvested, washed and added to the coated plates in the presence or absence of SLK-peptide, CLK-peptide, or control peptide, and allowed to incubate in 1.0% serum-containing medium. Cells are harvested, washed, and lysed in 1.0% Triton X-100 buffer containing 300 mM NaCl, 50 mM Tris (pH 7.0) and 1× protease inhibitor cocktail. Equal amounts (25 μg/lane) of tumor cell lysates are separated by SDS Page and transferred to nitrocellulose membranes. Membranes are probed by incubation with either polyclonal antibodies directed to P21^{CIP1} or Actin (Santa Cruz) as described previously (Brooks et al., Cell 1998, 92: 391-400; Petitclerc et al., Cancer Res. 1999, 59: 2724-2730). Western blots are visualized by a chemiluminesence detection system (Amersham Life Sciences).

Incubation of B16F10 tumor cells plated on denatured collagen type-IV with SLK-peptide or CLK-peptide results in a significant increase in expression of certain proteins and a significant decrease in expression of other proteins as compared to either no treatment or treatment with a control peptide. These findings indicate that tumor cell interactions with the HUIV26 cryptic epitope within collagen type-IV play a unique role in regulating expression of specific genes.

### Example XI

### Inhibition of Cellular Interactions with the HUI77 Cryptic Site Enhances Expression of P21^{CIP1} RNA

Differential cDNA array analysis of HUVECs treated with Mab HUI77 suggested a significant increase in the expression of several genes including that for cyclin dependent kinase inhibitor P21^{CIP1}.

Using the methods described in Example V, an Affymetrix™-based differential cDNA array analysis was performed. HUVECS treated or not treated with Mab HUI77 were used. RNA was isolated and used to synthesize double-stranded cDNA. The synthesized cDNA was converted to cRNA, and biotin-labeled, fragmented and hybridized to a U95Av2 chip.

Relative expression levels of P21^{CIP1} were then assessed by real time RT-PCR. HUVECs were allowed to interact with denatured collagen type-IV in the presence or absence of Mab HUI77 or an isotype-matched control antibody, and mRNA and whole cell lysates were prepared.

Real Time quantitative RT-PCR and real-time fluorescence detection were carried out as described in Example V, and the same primer sets were used to detect P21^{CIP1} mRNA and control gene β2-macroglobulin.

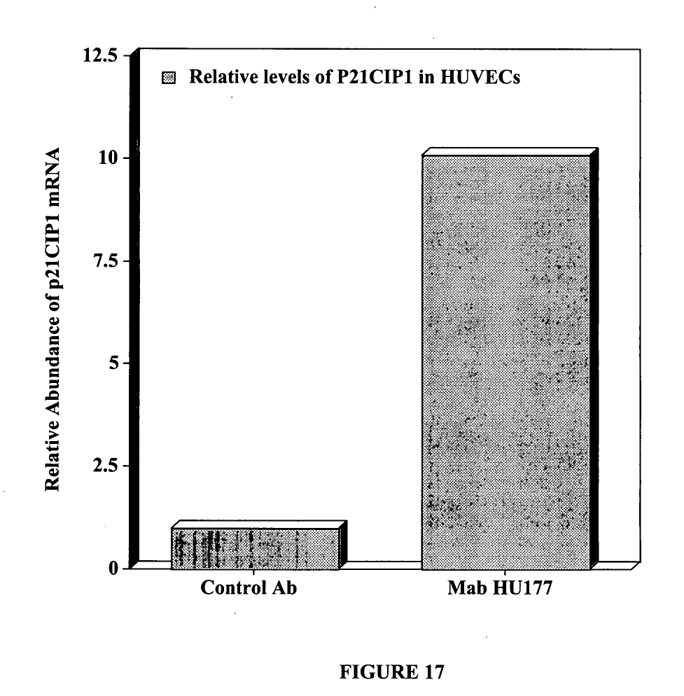

As shown in FIG. 17, the relative level of P21^{CIP1} mRNA was increased by approximately 10-fold as compared to an isotype-matched non-specific control antibody. Moreover, no changes in the relative levels of control genes β-actin or β2-macroglobulin were observed following treatment of HUVECs with Mab HUI77 (data not shown).

### Example XII

### Inhibition of Cellular Interactions with the HUI77 Cryptic Site Enhances Expression of P27^{KIP1} RNA

Differential cDNA array analysis of HUVECs treated with Mab HUI77 suggested a significant increase in the expression of several genes including that for cyclin-dependent kinase inhibitor P27^{KIP1}.

As described in Example V, an Affymetrix™-based differential cDNA array analysis was performed. HUVECS treated or not treated with Mab HUI77 were used. As described above, RNA was isolated and utilized to synthesize double-stranded cDNA. The synthesized cDNA was converted to cRNA, and biotin-labeled, fragmented and hybridized to a U95Av2 chip.

Relative expression levels of P27^{KIP1} were then assessed by real time RT-PCR. HUVECs were allowed to interact with denatured collagen type-IV in the presence or absence of Mab HUI77 or an isotype-matched control antibody, and mRNA and whole cell lysates were prepared.

Real Time quantitative RT-PCR and real-time fluorescence detection were carried out as described in Example V.

The primer sets used to detect p27^{KIP1} mRNA were:

| | |
|---|---|
| 5′-TgCAACCgACgATTCTTCTA-3′ | (forward; SEQ ID NO: 11) |
| and | |
| 5′-CgAgCTgTTTACgTTTgACg-3′. | (reverse; SEQ ID NO: 12) |

The primers used to detect control gene β2-macroglobulin were:

SEQ ID NO: 3)

5′-AAAGATGAGTATGCCTGCCG-3′ (forward;
and

SEQ ID NO: 4)

5′-CCTCCATGATGATGCTGCTTACA-3′ (reverse;.

As shown in FIG. 18, the relative level of P27^{KIP1} mRNA was increased by greater than 5-fold as compared to an isotype-matched non-specific control antibody. Moreover, no changes in the relative levels of control genes β-actin or β2-macroglobulin were observed following treatment of HUVECs with Mab HUI77 (data not shown).

### Example XIII

### Inhibition of Cellular Interactions by CLK-Peptide Enhances Expression of P27^{KIP1}

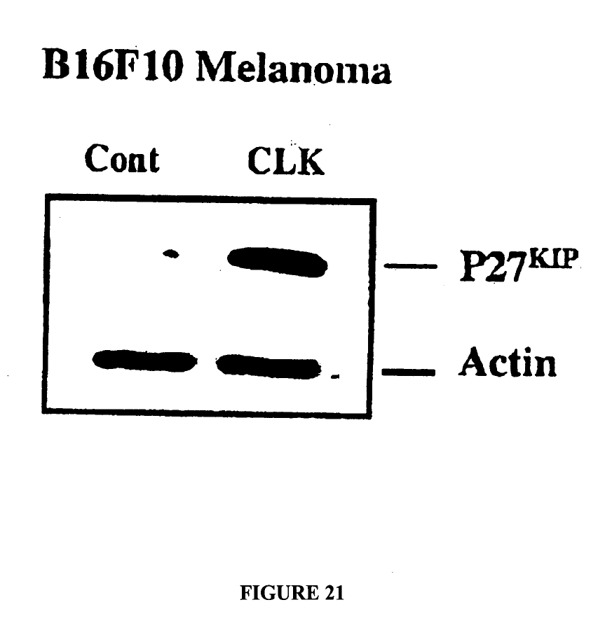

To examine the effect of CLK-peptide on the expression of cyclin-dependent kinase P27^{KIP1}, B16F10 melanoma and GL261 glioblastoma cells were resuspended in adhesion buffer in the presence or absence of CLK-peptide or control peptide. Cells were added to culture plates coated with denatured collagen and allowed to incubate for 24 hours. Total cell lysates were prepared and the proteins analyzed by Western Blot analysis. As shown in FIG. 21, treatment of either cell type with CLK-peptide caused a significant upregulation of P27^{KIP1}. These findings suggest that CLK-peptide may affect tumor cell proliferation by up-regulating the CDK inhibitor P27^{KIP1}.

### Example XIV

### Inhibition of Cellular Interactions by CLK-Peptide Enhances Expression of P21^{CIP1}

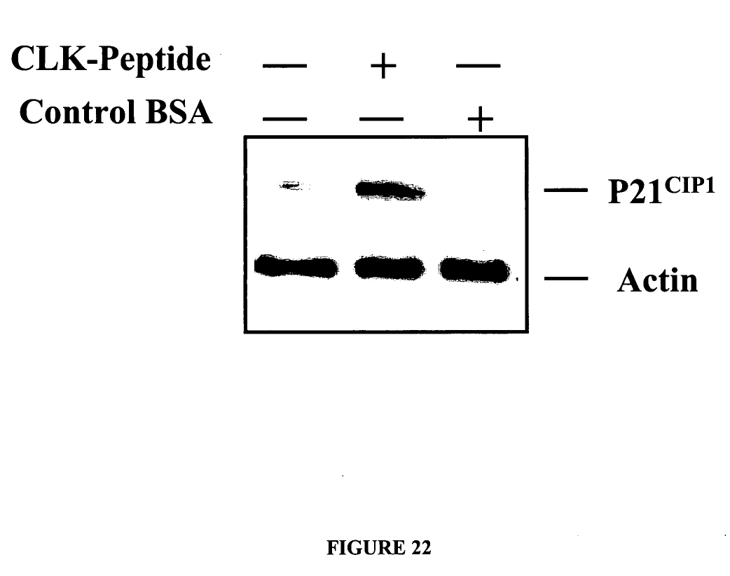

The effect of CLK-peptide on the expression of P21^{CIP1} was examined by Western Blot analysis. M21 cells were resuspended in adhesion buffer in the presence or absence of CLK-peptide and allowed to incubate for 12 hours in 1% serum-containing medium. Total cell lysates were prepared and the levels of P21^{CIP1} and actin analyzed by Western Blot analysis. As shown in FIG. 22, treatment with CLK-peptide caused a significant upregulation of P21^{CIP1}.

What is claimed is:

1. A method for identifying at least one gene or protein, wherein the expression of said gene or protein is modulated by binding of an antagonist to a cryptic epitope of an ECM component, wherein said antagonist specifically binds to said cryptic epitope of said ECM component, comprising the steps of:
a) treating cells with the antagonist;
b) measuring gene expression or protein levels in the cells;
c) comparing said gene expression or protein levels with control gene expression or protein levels measured in cells not treated with the antagonist, and;
d) identifying a gene or protein in the cells wherein levels in the cells treated with the antagonist are modulated as compared to control cell gene expression or protein levels.

2. The method of claim 1 wherein at least two genes or proteins are identified in the method of identifying, and wherein one of the at least two genes or proteins identified is IGFBP-4, TSP-1, Id-1, p27^{KIP} or p21^{CIP}.

3. The method of claim 1 wherein the antagonist is an antibody or an antibody fragment, or a peptide.

4. The method of claim 3 wherein the antibody is a monoclonal antibody or a polyclonal antibody.

5. The method of claim 4 wherein the antagonist is monoclonal antibody HUIV26.

6. The antagonist of claim 3 wherein the antagonist is a CLK-peptide, a SLK-peptide, KGGCLK-peptide (SEQ ID NO: 13), the peptide NH₂—S-T-Q-N-A-S-L-L-S-L-T-V—C—COOH (SEQ ID NO: 14), STQ-peptide, or STQ-peptide-S.

7. A method for inhibiting tumor metastasis, cell adhesion, cell migration, tumor growth, angiogenesis, or for treating an angiogenesis-dependent condition, comprising administering a product of a gene, or administering a protein, wherein the gene or the protein is modulated by the binding of an antagonist to a cryptic epitope of an ECM component, and wherein said antagonist specifically binds to said cryptic epitope of said ECM component, and wherein the gene or the protein is identified using a method of identifying at least one gene or protein modulated by the binding of said antagonist to said epitope, said method of identifying comprising the steps of:
a) treating cells with the antagonist;
b) measuring gene expression or protein levels in the cells;
c) comparing said gene expression or protein levels with control gene expression or protein levels measured in cells not treated with the antagonist, and;
d) identifying a gene or protein in the cells wherein levels of the gene or protein in the cells treated with the antagonist are modulated as compared to control cell gene expression or protein levels.

8. The method of claim 7 wherein said gene product or protein is administered in conjunction with chemotherapy, radiation therapy, or a cytostatic agent.

9. The method of claim 7 wherein at least two genes or proteins are identified in the method of identifying, and wherein one of the at least two genes or proteins identified is IGFBP-4, TSP-1, Id-1, p27^{KIP} or p21^{CIP}.

10. The method of claim 7 wherein the antagonist is an antibody or an antibody fragment, or a peptide.

11. The method of claim 10 wherein the antibody is a monoclonal antibody or a polyclonal antibody.

12. The method of claim 11 wherein the antagonist is monoclonal antibody HUIV26.

13. The antagonist of claim 10 wherein the antagonist is a CLK-peptide, a SLK-peptide, KGGCLK-peptide (SEQ ID NO: 13), the peptide NH₂—S-T-Q-N-A-S-L-L-S-L-T-V—C—COOH (SEQ ID NO: 14), STQ-peptide, or STQ-peptide-S.

14. An antagonist that specifically binds to a cryptic epitope of an ECM component, wherein binding of said antagonist to said cryptic epitope of said ECM component results in modulation of IGFBP-4, TSP-1, Id-1, p27^{KIP} or p21^{CIP}.

15. The antagonist of claim 14 wherein the antagonist is an antibody or an antibody fragment, or a peptide.

16. The antagonist of claim 15 wherein the antagonist is a monoclonal antibody or a polyclonal antibody.

17. The antagonist of claim 16 wherein the antagonist is monoclonal antibody HUIV26.

18. The antagonist of claim 15 wherein the antagonist is CLK-peptide, SLK-peptide, KGGCLK-peptide (SEQ ID NO: 13), the peptide NH₂—S-T-Q-N-A-S-L-L-S-L-T-V—C—COOH (SEQ ID NO: 13), STQ-peptide, or STQ-peptide-S.

19. A method of inhibiting tumor metastasis, cell adhesion, cell migration, tumor growth, angiogenesis, or treating an angiogenesis-dependent condition, comprising administering the antagonist of claim 14.

20. The method of claim 19 wherein said antagonist is administered in conjunction with chemotherapy, radiation therapy, or a cytostatic agent.

21. A method of detecting the inhibition of tumor metastasis, cell adhesion, cell migration, tumor growth, or angiogenesis, using an antagonist that specifically binds to a cryptic epitope of an ECM component, comprising: measuring the level of IGFBP-4, TSP-1, Id-1, or p21^{CIP}, wherein said level of IGFBP-4, TSP-1, Id-1, p27^{KIP} or p21^{CIP} is modulated.
